(19) United States
(12) Patent Application Publication
(10) Pub. No.: US 2009/0210026 A1
(43) Pub. Date: Aug. 20, 2009

(54) SPG STIMULATION FOR ENHANCING NEUROGENESIS AND BRAIN METABOLISM

(75) Inventors: Yoram Solberg, Herzeliya (IL); Gal Ariav, Givaat-Ada (IL); Hernan Altman, Haifa (IL); Avinoam Dayan, Zikron Yaakov (IL)

Correspondence Address: RATNERPRESTIA, P.O. BOX 980, VALLEY FORGE, PA, 19482, US

(73) Assignee: BRAINSGATE LTD., Caesarea (IL)

(21) Appl. No.: 12/197,614

(22) Filed: Aug. 25, 2008

Related U.S. Application Data
(63) Continuation-in-part of application No. 11/465,381, filed on Aug. 17, 2006.
(60) Provisional application No. 60/966,613, filed on Aug. 28, 2007.
(60) Provisional application No. 60/966,614, filed on Aug. 28, 2007.

(51) Int. Cl. A61N 1/36

(52) U.S. Cl. 607/45

(57) ABSTRACT

A method is provided, including identifying an electrical stimulation protocol as being suitable for augmenting genesis of one or more cell populations in at least one brain region of the subject. The cell genesis is augmented by applying the identified stimulation protocol to an SPG, a greater palatine nerve, a branch of the greater palatine nerve, a lesser palatine nerve, a sphenopalatine nerve, a communicating branch between a maxillary nerve and an SPG, an otic ganglion, an afferent fiber going into the otic ganglion, an efferent fiber going out of the otic ganglion, an infraorbital nerve, a vidian nerve, a greater superficial petrosal nerve, a lesser deep petrosal nerve, a maxillary nerve, a branch of the maxillary nerve, a nasopalatine nerve, a peripheral site that provides direct or indirect afferent innervation to the SPG, or a peripheral site that is directly or indirectly efferently innervated by the SPG.

### CROSS-REFERENCE TO RELATED APPLICATIONS

The present application is a continuation-in-part of U.S. application Ser. No. 11/465,381, filed Aug. 17, 2006, which is assigned to the assignee of the present application and is incorporated herein by reference.

The present application claims the benefit of: (i) U.S. Provisional Application 60/966,613, filed Aug. 28, 2007, and (ii) U.S. Provisional Application 60/966,614, filed Aug. 28, 2007, both of which are assigned to the assignee of the present application and are incorporated herein by reference.

### FIELD OF THE INVENTION

The present invention relates generally to medical procedures and devices. More specifically, the invention relates to the use of stimulation for treating medical conditions of the brain.

### BACKGROUND OF THE INVENTION

Stimulation of the sphenopalatine ganglion (SPG) treats various acute brain hypoperfusion states, such as occur during acute ischemic stroke. During acute ischemic stroke, blood supply to the brain is abruptly blocked by either a thrombus or an embolus. As a result, the blood supply to a localized area of the brain in the center of the infarction is substantially reduced to a level that is insufficient to meet the tissue's metabolic needs for oxygen and other nutrients. This reduction leads to rapid neuronal cell death in this area within several minutes after the stroke. The blood supply received by adjacent tissue, called the penumbra, is insufficient to main the metabolism of this tissue, which thus suffers from hypoxic conditions which leads to gradual neuronal death within several hours after the stroke. Following stroke, new neurons are generated, which migrate to the infarcted areas in an attempt to restore the damaged tissue.

US Patent Application Publication 2007/0083245 to Lamensdorf et al., which is incorporated herein by reference, describes apparatus for treatment, including one or more electrodes, configured to be applied to a site of a subject, and adverse cerebrovascular condition treatment functionality. The functionality comprises a control unit configured to drive the one or more electrodes to apply electrical stimulation to the site during a plurality of stimulation periods which includes at least first and last stimulation periods, set an inter-period interval between initiation of the first stimulation period and initiation of the last stimulation period to be at least 24 hours, and configure the stimulation during the first and last stimulation periods to induce at least one neuroprotective occurrence selected from the group consisting of: an increase in cerebral blood flow (CBF) of the subject, and a release of one or more neuroprotective substances. The site is selected from the group consisting of: a sphenopalatine ganglion (SPG), a greater palatine nerve, a lesser palatine nerve, a sphenopalatine nerve, a communicating branch between a maxillary nerve and an SPG, an otic ganglion, an afferent fiber going into the otic ganglion, an efferent fiber going out of the otic ganglion, an infraorbital nerve, a vidian nerve, a greater superficial petrosal nerve, and a lesser deep petrosal nerve.

The '245 publication describes an in vivo experiment assessing the effect of long-term rehabilitative SPG stimulation. A rat tMCAO model of stroke was used to evaluate the benefits, including neuromuscular, motility, cognitive, somatosensory, somatomotor, infarct volume benefits, of rehabilitative SPG stimulation using the techniques described therein. The stimulation was applied for seven consecutive days beginning at 24 hours after reperfusion in the tMCAO model. In summary, in the experiment, SPG stimulation initiated 24 hours after tMCAO had advantageous results for all five parameter groups evaluated. In addition, SPG stimulation increased the number of neurons in all regions counted.

PCT Publication WO 01/85094 and US Patent Application Publications 2004/0015068 and 2004/0210269 to Shalev and Gross, which are incorporated herein by reference, describe apparatus for modifying a property of a brain of a patient, including electrodes applied to a sphenopalatine ganglion (SPG) or a neural tract originating in or leading to the SPG. A control unit drives the electrodes to apply a current capable of inducing (a) an increase in permeability of a blood-brain barrier (BBB) of the patient, (b) a change in cerebral blood flow of the patient, and/or (c) an inhibition of parasympathetic activity of the SPG.

U.S. Pat. No. 7,117,033 to Shalev et al., which is incorporated herein by reference, describes a method for treating a subject, including positioning at least one electrode at least one site of the subject, such as the SPG, for less than about 3 hours, applying an electrical current to the site of the subject, and configuring the current to increase cerebral blood flow (CBF) of the subject, so as to treat a condition of the subject.

PCT Publication WO 04/043218 to Gross et al., which is incorporated herein by reference, describes apparatus for treating a subject, including (a) a stimulation device, adapted to be implanted in a vicinity of a site selected from the list consisting of: a SPG and a neural tract originating in or leading to the SPG; and (b) a connecting element, coupled to the stimulation device, and adapted to be passed through at least a portion of a greater palatine canal of the subject.

U.S. Pat. No. 6,526,318 to Ansarinia and related PCT Publication WO 01/97905 to Ansarinia, which are incorporated herein by reference, describe a method for the suppression or prevention of various medical conditions, including pain, movement disorders, autonomic disorders, and neuropsychiatric disorders. The method includes positioning an electrode on or proximate to at least one of the patient's SPG, sphenopalatine nerves, or vidian nerves, and activating the electrode to apply an electrical signal to such nerve. In a further embodiment for treating the same conditions, the electrode used is activated to dispense a medication solution or analgesic to such nerve.

The following patent application publications, all of which are incorporated herein by reference, may be of interest: WO 03/090599, WO 04/010923, WO 04/043218, WO 04/044947, WO 04/045242, WO 04/043217, WO 04/043334, WO 05/030025, WO 05/030118, US 2004/0220644, US 2006/0020299, and US 2005/0159790.

The following patents and patent application publications, all of which are incorporated herein by reference, may be of interest:

U.S. Pat. No. 6,853,858 to Shalev

U.S. Pat. No. 7,146,209 to Gross et al.

U.S. Pat. No. 5,752,515 to Jolesz et al.

U.S. Pat. No. 6,405,079 to Ansarinia

U.S. Pat. No. 6,432,986 to Levin

US Patent Application 2001/0004644 to Levin

Sun Y et al., in an article entitled, “Neuronal nitric oxide synthase and ischemia-induced neurogenesis,” J Cereb Blood Flow Metab 25(4):485-92 (2005), which is incorporated herein by reference, report that nitric oxide (NO) influences infarct size after focal cerebral ischemia and also regulates neurogenesis in the adult brain. These observations suggest that therapeutic approaches to stroke that target NO signaling may provide neuroprotection and also enhance brain repair through cell replacement. In addition, ischemia itself stimulates neurogenesis, and ischemia-induced neurogenesis may be regulated differently than neurogenesis in nonischemic brain. Selective inhibition of neuronal NO synthase may have the potential to both reduce infarct size and enhance neurogenesis in stroke.

Wilner A, in an article entitled, “Who is at risk for post-stroke dementia?” Neurology Reviews.com Vol. 11, No. 2 (February 2003), which is incorporated herein by reference, describes the results of a study analyzing risk factors for survival of a stroke and post-stroke dementia.

Hotta H et al., in an article entitled, “Effects of stimulating the nucleus basalis of Meynert on blood flow and delayed neuronal death following transient ischemia in rat cerebral cortes,” Jap J Phys 52:383-393 (2002), which is incorporated herein by reference, report that stimulation of the nucleus basalis of Meynert (NBM) in the rat was accompanied by vasodilatation and increase in cortical blood flow. They suggest that NBM-originating vasodilative activation can protect the ischemia-induced delayed death of cortical neurons by preventing a blood flow decrease in widespread cortices.

Reis D J et al., in an article entitled, “Electrical stimulation of cerebellar fastigial nucleus reduces ischemic infarction elicited by middle cerebral artery occlusion in rat,” J Cereb Blood Flow Metab 11(5):810-8 (1991), which is incorporated herein by reference, report that electrical stimulation of the cerebellar fastigial nucleus (FN) profoundly increases cerebral blood flow via a cholinergic mechanism. Utilizing the rat middle cerebral artery occlusion (MCAO) model, they demonstrated that one hour of electrical stimulation of the FN has the capacity to substantially reduce the infarct size at the rim of the cortex dorsal and ventral to the infarction, and medially within the thalamus and striatum corresponding to the penumbral zone. They conclude that excitation of an intrinsic system in brain represented in the rostral FN has the capacity to substantially reduce an ischemic infarction.

Matsui T et al., in an article entitled, “The effects of cervical spinal cord stimulation (cSCS) on experimental stroke,” Pacing Clin Electrophysiol 12(4 Pt 2):726-32 (1989), which is incorporated herein by reference, report that cSCS increases regional cerebral blood flow, and, in a cat middle cerebral artery occlusion model (MCAO), reduced the rate of death within 24 hours after MCAO.

Segher O et al., in an article entitled, “Spinal cord stimulation reducing infract volume in model of focal cerebral ischemia in rats,” J Neurosurg 99(1):131-137 (2003), which is incorporated herein by reference, demonstrate that spinal cord stimulation increases cerebral blood flow in rats and significantly reduces stroke volume, suggesting that spinal cord stimulation could be used for treatment and prevention of stroke.

The following references, which are incorporated herein by reference, may be of interest:
Henninger N et al., “Stimulating circle of Willis nerve fibers preserves the diffusion-perfusion mismatch in experimental stroke,” Stroke 2007; 38:2779-2786
Ayajiki K et al., “Effects of capsaicin and nitric oxide synthase inhibitor on increase in cerebral blood flow induced by sensory and parasympathetic nerve stimulation in the rat,” J Appl Physiol 2005; 98:1792-1798
Yarnitsky D et al., “Reversal of cerebral vasospasm by sphenopalatine ganglion stimulation in a dog model of subarachnoid hemorrhage,” Surg Neurol 2005; 64:5-11; discussion 11
Kano M et al., “Parasympathetic denervation of rat pial vessels significantly increases infarction volume following middle cerebral artery occlusion,” J Cereb Blood Flow Metab 1991; 11:628-637
Koketsu N et al., “Chronic parasympathetic sectioning decreases regional cerebral blood flow during hemorrhagic hypotension and increases infarct size after middle cerebral artery occlusion in spontaneously hypertensive rats,” J Cereb Blood Flow Metab 1992; 12:613-620
Weber R et al., “Present status of magnetic resonance imaging and spectroscopy in animal stroke models,” J Cereb Blood Flow Metab 2006; 26:591-604
Sager T N et al., “Changes in N-acetyl-aspartate content during focal and global brain ischemia of the rat,” J Cereb Blood Flow Metab 1995; 15:639-646
Moffett J R et al., “N-acetylaspartate in the CNS: From neurodiagnostics to neurobiology,” Progress in Neurobiology 2007; 81:89-131
Franke C et al., “Probability of metabolic tissue recovery after thrombolytic treatment of experimental stroke: a magnetic resonance spectroscopic imaging study in rat brain,” J Cereb Blood Flow Metab 2000; 20:583-591
Demougeot C et al., “N-Acetylaspartate, a marker of both cellular dysfunction and neuronal loss: its relevance to studies of acute brain injury,” J Neurochem 2001; 77:408-415.
Demougeot C et al., “Reversible loss of N-acetyl-aspartate in rats subjected to long-term focal cerebral ischemia,” J Cereb Blood Flow Metab 2003; 23:482-489.
Meyer J S et al., “Cognition and cerebral blood flow fluctuate together in multi-infarct dementia,” Stroke 1988; 19:163-169
Markus H S et al., “Reduced cerebral blood flow in white matter in ischemic leukoaraiosis demonstrated using quantitative exogenous contrast based perfusion MRI,” J Neurol Neurosurg Psychiatry 2000; 69:48-53
Kawamura J et al., “Leukoaraiosis correlates with cerebral hypoperfusion in vascular dementia,” Stroke 1991; 22:609-614
Schmidt R et al., “White matter lesion progression, brain atrophy, and cognitive decline: The Austrian Stroke Prevention Study,” Ann Neurol 2005; 58:610-616
Seylaz J et al., “Effects of stimulation of the sphenopalatine ganglion on cortical blood flow in the rat,” Journal of Cerebral Blood Flow and Metabolism,” 8, 875-878 (1988)
Suzuki N et al., “Selective electrical stimulation of postganglionic cerebrovascular parasympathetic nerve fibers originating from the sphenopalatine ganglion enhances cortical blood flow in the rat,” Journal of Cerebral Blood Flow and Metabolism, 10, 383-391 (1990)
Branston N M, “The physiology of the cerebrovascular parasympathetic innervation,” British Journal of Neurosurgery 9:319-329 (1995)
Branston N M et al., “Contribution of cerebrovascular parasympathetic and sensory innervation to the short-term control of blood flow in rat cerebral cortex,” J Cereb Blood Flow Metab 15(3):525-31 (1995)
Toda N et al., “Cerebral vasodilation induced by stimulation of the pterygopalatine ganglion and greater petrosal nerve in anesthetized monkeys,” Neuroscience 96(2):393-398 (2000)
Seylaz J et al., “Effect of stimulation of the sphenopalatine ganglion on cortical blood flow in the rat,” J Cereb Blood Flow Metab 8(6):875-8 (1988)
Ziche M et al., “Nitric oxide and angiogenesis,” J Neurooncol 50:139-148 (2000)
Kawamata T et al., “Intracisternal basic fibroblast growth factor (bFGF) enhances behavioral recovery following focal cerebral infarction in the rat,” J Cereb Blood Flow Metab 16:542-547 (1996)
Zhang F et al., “Nitric oxide donors increase blood flow and reduce brain damage in focal ischemia: evidence that nitric oxide is beneficial in the early stages of cerebral ischemia,” J Cereb Blood Flow Metab 14(2):217-26 (1994)
Beridze M et al., “Effect of nitric oxide initial blood levels on erythrocyte aggregability during 12 hours from ischemic stroke onset,” Clin Hemorheol Microcirc 30(3-4):403-6 (2004)
Phan T G et al., “Salvaging the ischaemic penumbra: more than just reperfusion?” Clin Exp Pharmacol Physiol 29(1-2):1-10 (2002)
Zhang R et al., “Nitric oxide enhances angiogenesis via the synthesis of vascular endothelial growth factor and cGMP after stroke in the rat,” Circ Res 21; 92(3):308-13 (2003)
Zhang R et al., “A nitric oxide donor induces neurogenesis and reduces functional deficits after stroke in rats,” Ann Neurol 50:602-611 (2001)
Ziche M et al., “Nitric oxide and angiogenesis,” J Neurooncol 50:139-148 (2000)
de la Torre J C, “Vascular basis of Alzheimer's pathogenesis,” Ann NY Acad Sci 977:196-215 (2002)
Sandgren K et al., “Vasoactive intestinal peptide and nitric oxide promote survival of adult rat myenteric neurons in culture,” J Neurosci Res 72(5):595-602 (2003)
Khan M et al., “S-Nitrosoglutathione reduces inflammation and protects brain against focal cerebral ischemia in a rat model of experimental stroke,” J Cereb Blood Flow Metab 25(2):177-92 (2005)
Zausinger V S et al., “Neurological impairment in rats after transient middle cerebral artery occlusion: a comparative study under various treatment paradigms,” Brain Research 863(1-2):94-105 (2000)

### SUMMARY OF THE INVENTION

In some embodiments of the present invention, excitatory electrical stimulation is applied to the sphenopalatine ganglion (SPG) to augment neurogenesis in a subject suffering from an adverse cerebral condition, such as cerebral infarction, in order to improve recovery from the condition. The inventors hypothesize that such stimulation may augment the neurogenesis by increasing blood perfusion to the damaged tissue, which improves the supply of oxygen and other nutrients to the tissue, and/or by causing the release of neurotransmitters and/or neuromodulators from SPG nerve fibers. Alternatively, the stimulation is applied to another “modulation target site” (MTS), as defined hereinbelow.

To evaluate the level of augmentation of neurogenesis in infarcted rat brains caused by SPG stimulation, the inventors conducted an experiment using a rat middle cerebral artery occlusion (MCAO) model of stroke. Stroke was induced in two rats, one of which served as a control, and the other of which was treated with SPG stimulation beginning 24 hours after stroke. The rats were sacrificed, and histopathological examinations and immunohistochemical analysis for markers of newly born cells were performed. The examinations indicated that the SPG-stimulated rat had substantially more newly born cells than the control rat, and that a substantially greater percentage of the newly born cells were neuroblasts (the progenitor cells of neurons) in the SPG-stimulated rat than in the control rat.

In some embodiments of the present invention, stimulation of the SPG or another MTS augments neurogenesis in an infarcted brain, thereby leading to a better prognosis for acute stroke patients. Alternatively, such stimulation augments cell genesis (neurogenesis, angiogenesis, and/or glia-genesis) in a brain suffering from another adverse cerebral condition, such as chronic cerebral hypoperfusion states (such as occur in vascular dementia and Alzheimer's disease), neurodegenerative disorders (such as Parkinson's disease) and electrical hyper-activity states (such as epilepsy).

In some embodiments of the present invention, a method is provided that comprises identifying that a subject may benefit from augmented neurogenesis, and, responsively to the identifying, applying stimulation to the SPG or another MTS of the subject. Alternatively or additionally, the method comprises identifying a stimulation protocol as suitable for augmenting neurogenesis, and applying the identified protocol to a subject who may benefit from augmented neurogenesis.

In some embodiments of the present invention, a method is provided for augmenting neurogenesis in a subject who has not been diagnosed with any neurological condition associated with reduced neurogenesis or an elevated level of brain cell death. The method comprises identifying that the subject may benefit from augmented neurogenesis, and, responsively to identifying, applying chronic, long-term stimulation to an MTS, such as the SPG. The chronic stimulation has a duration of at least two weeks, at least four weeks, at least three months, at least six months, or the remaining life of the subject. During this chronic treatment, stimulation is typically applied intermittently, such as during one session per day, or less frequently, such as depending on the severity of assessed risk. Alternatively, the stimulation is applied generally constantly, typically at a low strength.

For some applications, identifying that the subject may benefit from augmented neurogenesis comprises identifying that the subject is at least a threshold age. For example, the threshold age may be between about 50 and about 80 years. Alternatively or additionally, the subject may identified because the subject suffers from one or more symptoms of a vascular disorder, such as hypertension, peripheral vascular disease, or coronary artery disease. Alternatively or additionally, the method comprises identifying that the subject suffers from reduced cardiac output, e.g., caused by heart failure. Further alternatively or additionally, the subject may be identified as potentially benefiting from augmented neurogenesis if the subject has a family history of any type of dementia, and/or the subject has radiological findings suggestive of cerebral vascular disease or Alzheimer's disease.

The stimulation is typically applied on a chronic, long-term basis, i.e., for at least one week, such as at least two weeks, at least four weeks, at least three months, at least six months, or during the remaining life of the subject. During this chronic treatment, stimulation is typically applied intermittently, such as during one session per day, or less frequently, such as depending on the severity of assessed risk. Alternatively, the stimulation is applied generally constantly, typically at a low strength. For some applications, the stimulation is applied bilaterally to both SPGs, while for other applications, the stimulation is applied unilaterally, such as to the MTS (e.g., the SPG) that supplies the more affected hemisphere of the brain.

In some embodiments of the present invention, excitatory electrical stimulation is applied to the SPG or another MTS of a subject who suffers from an adverse cerebral condition, in order to improve a metabolic state of a brain area affected by the adverse cerebral condition, thereby improving recovery from the condition. For some applications, the adverse cerebral condition is a cerebrovascular infarction, and the stimulation augments the recovery of the metabolic state of the brain area in the infarction or a vicinity of the infarction. The inventors hypothesize that such stimulation may improve the metabolic state by increasing blood perfusion to the damaged tissue, which improves the supply of oxygen and other nutrients to the tissue, and/or increases washout or otherwise reduces concentrations of toxic waste products from the damaged tissue. For example, lactate is a toxic waste product which, in high concentrations, leads to acidosis of the tissue.

The inventors conducted an experiment to assess the ability of SPG stimulation to augment stroke recovery in MCAO rats. Stroke was induced in twenty rats, of which 7 served as a control group, and of which 6 rats were treated with SPG stimulation beginning 18±2 h after stroke. Longitudinal ¹H magnetic resonance spectroscopic imaging (¹HMRSI) and diffusion MRI (DWI) were used to evaluate ischemic brain condition of the stimulated and control rats at 16±2 hours, 8 days, and 28 days after stroke. The inventors found that levels of N-Acetyl-Aspartate (NAA), which is considered to be a marker for neuronal density and viability levels, in the stimulated and control rats were the same 16±2 hours after stroke. 28 days after stroke, NAA levels were significantly higher in the stimulated group compared to the control group. This effect was more pronounced for regions with low baseline NAA values. In addition, a damage index, calculated based on DWI, showed significant deterioration for the controls which was not observed for the stimulated animals.

In some embodiments of the present invention, electrical stimulation of the SPG or another MTS reduces lactate concentration in the acute phase of a cerebrovascular infarction, or during another adverse cerebral condition. Such a reduction in lactate concentration results in a better metabolic state for the surviving cells. Preliminary experimental results in a rat model indicate the SPG stimulation reduces such lactate concentration in the acute phase of the infarction.

In some embodiments of the present invention, stimulation of the SPG or another MTS augments the recovery of the metabolic state of an infarcted brain area, which leads to a better prognosis for acute stroke patients. Alternatively, such stimulation augments the recovery of the metabolic state of a brain area affected by another adverse cerebral condition, such as chronic cerebral hypoperfusion states (such as occur in vascular dementia and Alzheimer's disease), neurodegenerative disorders (such as Parkinson's disease), and electrical hyperactivity states (such as epilepsy where there is a higher metabolic demand in the affected brain areas).

In some embodiments of the present invention, a method is provided for improving a metabolic state of the brain in a subject who has not been diagnosed with any neurological condition associated with a reduced level of brain metabolism. The method comprises identifying that the subject may benefit from an improved metabolic state, and, responsively to identifying, applying chronic, long-term stimulation to an MTS, such as the SPG. The chronic stimulation has a duration of at least two weeks, at least four weeks, at least three months, at least six months, or the remaining life of the subject. During this chronic treatment, stimulation is typically applied intermittently, such as during one session per day, or less frequently, such as depending on the severity of assessed risk. Alternatively, the stimulation is applied generally constantly, typically at a low strength.

The stimulation is typically applied on a chronic, long-term basis, i.e., for at least one week, such as at least two weeks, at least four weeks, at least three months, at least six months, or during the remaining life of the subject. During this chronic treatment, stimulation is typically applied intermittently, such as during one session per day, or less frequently, such as depending on the severity of assessed risk. Alternatively, the stimulation is applied generally constantly, typically at a low strength. For some applications, the stimulation is applied bilaterally to both SPGs, while for other applications, the stimulation is applied unilaterally, such as to the MTS (e.g., the SPG) that supplies the more affected hemisphere of the brain.

In the present patent application, a “modulation target site” (MTS) consists of:
an SPG (also called a pterygopalatine ganglion);
a nerve of the pterygoid canal (also called a vidian nerve), such as a greater superficial petrosal nerve (a preganglionic parasympathetic nerve) or a lesser deep petrosal nerve (a postganglionic sympathetic nerve);
a greater palatine nerve;
a branch of the greater palatine nerve
a lesser palatine nerve;
a sphenopalatine nerve;
a communicating branch between the maxillary nerve and the sphenopalatine ganglion;
an otic ganglion;
an afferent fiber going into the otic ganglion;
an efferent fiber going out of the otic ganglion;
an infraorbital nerve;
a maxillary nerve;
a branch of the maxillary nerve;
a nasopalatine nerve;
a peripheral (i.e., non-brain) site that provides direct or indirect afferent innervation to the SPG; or
a peripheral site that is directly or indirectly efferently innervated by the SPG (which causes retrograde activation of efferent nerve fibers, thereby stimulating the SPG).

For example, such peripheral sites may include mucosa of the nose or nasal pharynx, mucosa of the hard and soft palate, conjunctiva of the eye, and the lacrimal gland.

It is to be appreciated that references herein to specific modulation target sites are to be understood as including other modulation target sites, as appropriate.

It is further to be appreciated that insertion and modulation sites, methods of insertion and/or implantation, and parameters of modulation are described herein by way of illustration and not limitation, and that the scope of the present invention includes other possibilities which would be obvious to someone of ordinary skill in the art who has read the present patent application.

It is yet further to be appreciated that while some embodiments of the invention are generally described herein with respect to electrical transmission of power and electrical modulation of tissue, other modes of energy transport may be used as well. Such energy includes, but is not limited to, direct or induced electromagnetic energy, radiofrequency (RF) transmission, mechanical vibration, ultrasonic transmission, optical power, and low power laser energy (via, for example, a fiber optic cable).

It is additionally to be appreciated that whereas some embodiments of the present invention are described with respect to application of electrical currents to tissue, this is to be understood in the context of the present patent application and in the claims as being substantially equivalent to applying an electrical field, e.g., by creating a voltage drop between two electrodes.

In embodiments of the present invention, treating an adverse brain event or condition typically includes identifying that a subject is suffering from, and/or has suffered from, the brain event or condition.

There is therefore provided, in accordance with an embodiment of the present invention, a method including:

identifying an electrical stimulation protocol as being suitable for augmenting genesis of one or more cell populations in at least one brain region of the subject; and

augmenting the cell genesis by applying the identified stimulation protocol to a site of the subject selected from the group consisting of: a sphenopalatine ganglion (SPG), a greater palatine nerve, a branch of the greater palatine nerve, a lesser palatine nerve, a sphenopalatine nerve, a communicating branch between a maxillary nerve and an SPG, an otic ganglion, an afferent fiber going into the otic ganglion, an efferent fiber going out of the otic ganglion, an infraorbital nerve, a vidian nerve, a greater superficial petrosal nerve, a lesser deep petrosal nerve, a maxillary nerve, a branch of the maxillary nerve, a nasopalatine nerve, a peripheral site that provides direct or indirect afferent innervation to the SPG, and a peripheral site that is directly or indirectly efferently innervated by the SPG.

For some applications, the site is selected from the group consisting of: the SPG, the greater palatine nerve, the lesser palatine nerve, the sphenopalatine nerve, the communicating branch between the maxillary nerve and the SPG, the otic ganglion, the afferent fiber going into the otic ganglion, the efferent fiber going out of the otic ganglion, the infraorbital nerve, the vidian nerve, the greater superficial petrosal nerve, and the lesser deep petrosal nerve.

For some applications, identifying the stimulation protocol includes identifying the stimulation protocol as being suitable for augmenting neurogenesis in the at least one brain region. Alternatively or additionally, identifying the stimulation protocol includes identifying the stimulation protocol as being suitable for augmenting angiogenesis in the at least one brain region. Further alternatively or additionally, identifying the stimulation protocol includes identifying the stimulation protocol as being suitable for augmenting glia-genesis in the at least one brain region.

In an embodiment, the method includes identifying that the subject suffers from an adverse cerebral condition, and augmenting the cell genesis includes augmenting the cell genesis responsively to identifying that the subject suffers from the adverse cerebral condition. For some applications, the cerebral condition causes a brain area of the subject to be diseased, and the at least one brain region is selected from the group consisting of: a vicinity of the diseased brain area, and a brain area other than the vicinity of the diseased brain area. For some applications, identifying that the subject suffers from the adverse cerebral condition includes identifying that the subject suffers from a cerebrovascular infarction. Augmenting the cell genesis may include commencing applying the stimulation at least 18 hours after an occurrence of the infarction.

Typically, the method includes identifying that the subject may benefit from the augmented cell genesis, and augmenting the cell genesis includes augmenting the cell genesis responsively to identifying that the subject may benefit from the augmented cell genesis.

There is further provided, in accordance with an embodiment of the present invention, a method for treating a subject, including:

identifying an electrical stimulation protocol as being suitable for improving a metabolic state of a brain area of a subject; and

improving the metabolic state by applying the stimulation protocol to a site of the subject selected from the group consisting of: a sphenopalatine ganglion (SPG), a greater palatine nerve, a branch of the greater palatine nerve, a lesser palatine nerve, a sphenopalatine nerve, a communicating branch between a maxillary nerve and an SPG, an otic ganglion, an afferent fiber going into the otic ganglion, an efferent fiber going out of the otic ganglion, an infraorbital nerve, a vidian nerve, a greater superficial petrosal nerve, a lesser deep petrosal nerve, a maxillary nerve, a branch of the maxillary nerve, a nasopalatine nerve, a peripheral site that provides direct or indirect afferent innervation to the SPG, and a peripheral site that is directly or indirectly efferently innervated by the SPG.

For some applications, the site is selected from the group consisting of: the SPG, the greater palatine nerve, the lesser palatine nerve, the sphenopalatine nerve, the communicating branch between the maxillary nerve and the SPG, the otic ganglion, the afferent fiber going into the otic ganglion, the efferent fiber going out of the otic ganglion, the infraorbital nerve, the vidian nerve, the greater superficial petrosal nerve, and the lesser deep petrosal nerve.

In an embodiment, identifying the stimulation protocol includes identifying the stimulation protocol as suitable for reducing a lactate concentration in the brain area.

For some applications, the method includes identifying that the subject suffers from an adverse cerebral condition, and improving the metabolic state includes improving the metabolic state responsively to identifying that the subject suffers from the adverse cerebral condition. For some applications, identifying that the subject suffers from the adverse cerebral condition includes identifying that the subject suffers from a cerebrovascular infarction. For some applications, improving the metabolic state includes commencing applying the stimulation at least 18 hours after an occurrence of the infarction. For some applications, the brain area is an ischemic core of the infarction. Identifying the stimulation protocol may include identifying the stimulation protocol as suitable for reviving at least a portion of the ischemic core. For some applications, the brain area is an ischemic penumbra of the infarction. Identifying the stimulation protocol may include identifying the stimulation protocol as suitable for reviving at least a portion of the ischemic penumbra.

Typically, the method includes identifying that the subject may benefit from the improved metabolic state, and improving the metabolic state includes improving the metabolic state responsively to identifying that the subject may benefit from the improved metabolic state.

The present invention will be more fully understood from the following detailed description of the embodiments thereof, taken together with the drawings, in which:

### BRIEF DESCRIPTION OF THE DRAWINGS

FIG. 1 is a schematic illustration of a neural stimulation system, in accordance with an embodiment of the present invention;

FIGS. 2A-H are graphs showing results of an in vivo experiment assessing the effect of long-term rehabilitative SPG stimulation, measured in accordance with an embodiment of the present invention;

FIG. 3 is a graph showing changes in total normalized N-Acetyl-Aspartate (NAA) values at three experimental time points post-t-MCAO during a rat experiment performed in accordance with an embodiment of the present invention;

FIGS. 4A-C are graphs showing changes in NAA levels in respective brain areas categorized according to initial normalized NAA values measured in the experiment of FIG. 3;

FIG. 5 is a graph showing a damage index measured in the experiment of FIG. 3;

FIG. 6 is a graph showing average modified Neuro Severity Scores (mNSS) measured in the experiment of FIG. 3; and

FIGS. 7A-B and 8A-B show magnetic resonance spectra obtained during an experiment conducted by the inventors, measured in accordance with an embodiment of the present invention.

### DETAILED DESCRIPTION OF EMBODIMENTS

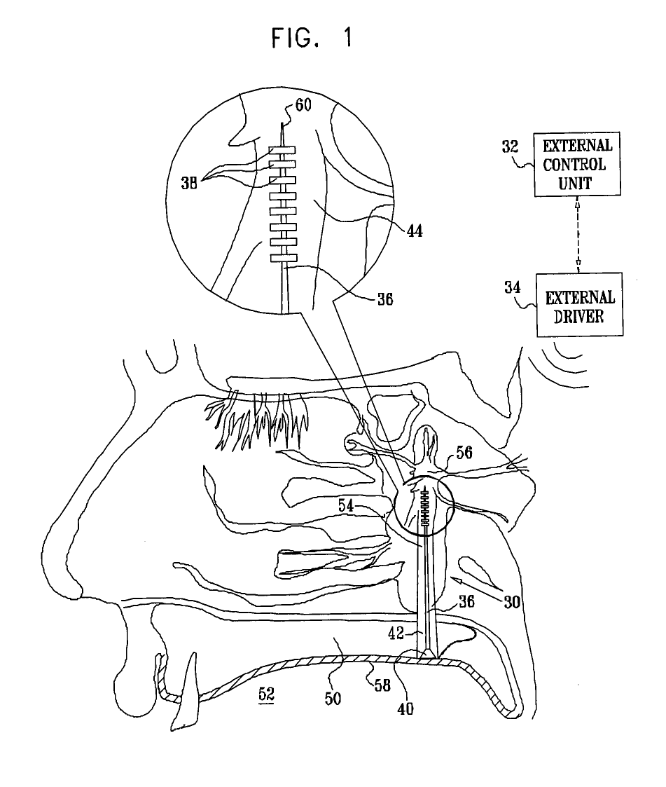

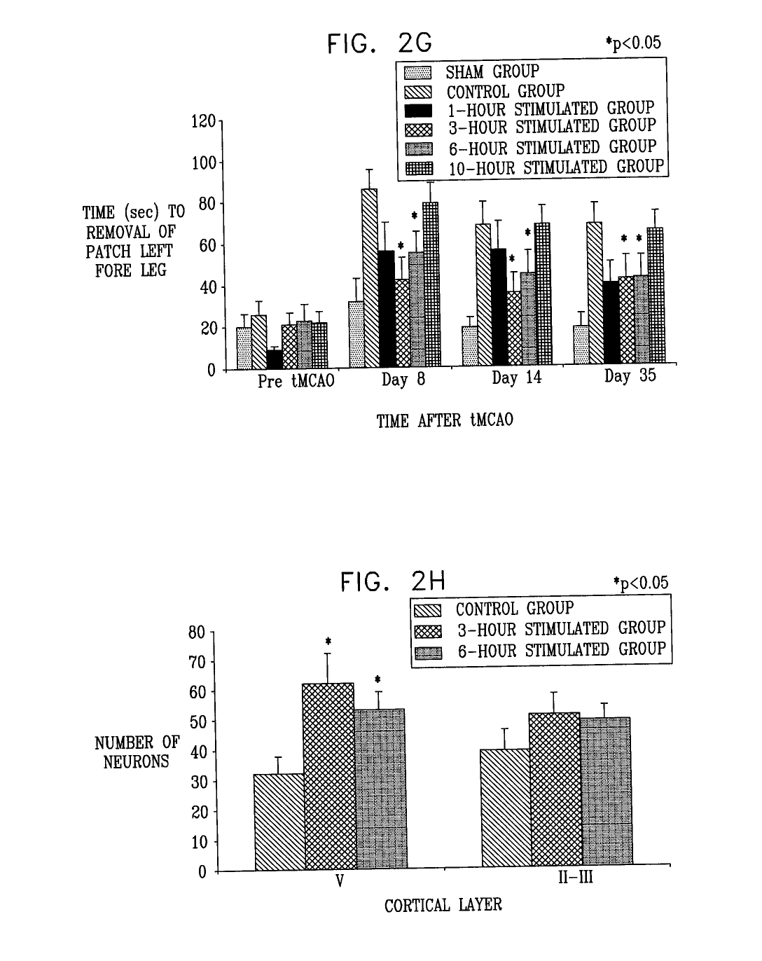

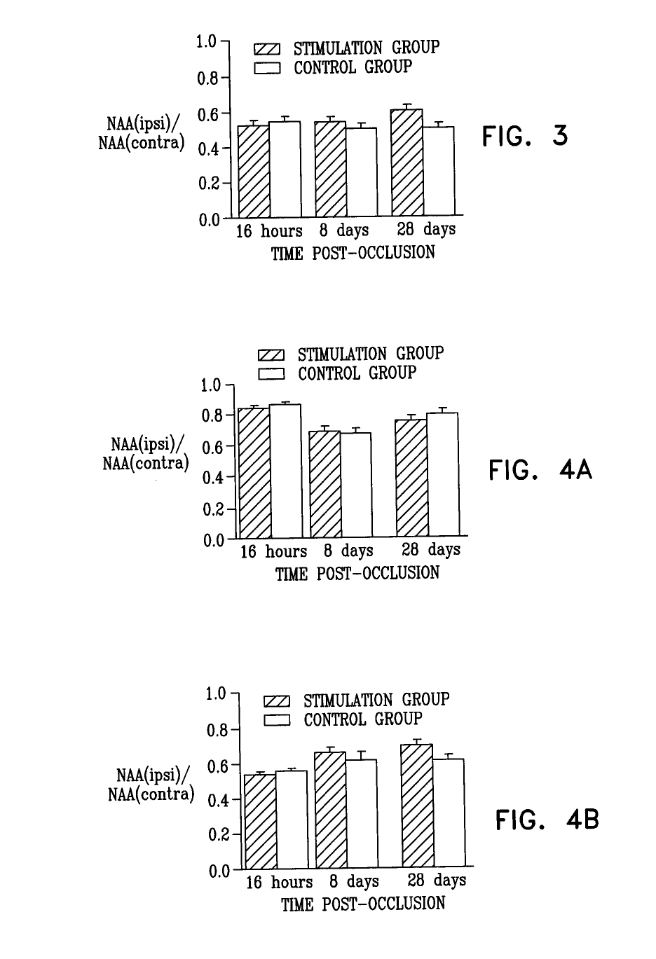

FIG. 1 is a schematic illustration of a neural stimulation system 20, in accordance with an embodiment of the present invention. System 20 typically comprises an implantable neural stimulator 30, an external control unit 32, and, for some applications, an external driver 34. Stimulator 30 comprises an elongated support element 36, one or more electrodes 38 fixed to the support element in a vicinity of a distal end thereof, and circuitry 40 coupled to the support element in a vicinity of a proximal end thereof. Circuitry 40 typically comprises a wireless coupling element (which typically comprises a coil, and additional elements, such as one or more rectifiers, capacitors, amplifiers, or filters. One or more leads (not shown in FIG. 1), which pass along, through, or around support element 36, couple electrodes 38 to circuitry 40. Alternatively, the leads function as the support element, i.e., the support element does not comprise any structural elements in addition to the leads. Circuitry 40 is shown schematically in FIG. 1; the circuitry may employ one or more of the more detailed configurations described with reference to FIGS. 3A-B, 4A-B, and 5A-D of U.S. patent application Ser. No. 11/349,020, filed Feb. 7, 2006, which published as US Patent Application Publication 2006/0287677, and which is incorporated herein by reference.

Stimulator 30 is configured to be passed through a greater palatine foramen 42 of a hard palate 50 of an oral cavity 52 of a subject into a greater palatine canal 54, such that electrodes 38 are brought into a vicinity of a sphenopalatine ganglion (SPG) 56. For some applications, the entire stimulator is contained within greater palatine canal 54, while for other applications, at least a portion of the circuitry and/or the support element are positioned submucosally in the oral cavity. For clarity of illustration, the greater and lesser palatine nerves, and the greater and less palatine arteries are not shown in the figures. During an implantation procedure, stimulator 30 is typically passed through greater palatine foramen 42 posterior to the greater palatine nerve and artery, which are manipulated into an anterior position within the canal.

For some applications, electrodes 38 apply a monophasic waveform to SPG 56, while for other applications, electrodes 38 apply a biphasic waveform. Alternatively or additionally, waveforms and/or stimulation techniques may be used that are described in one or more of the patent applications incorporated by reference hereinbelow, or waveforms and/or stimulation techniques may be used that are known in the art of neural stimulation.

Circuitry 40 of stimulator 30 comprises a stimulator wireless coupling element, which typically comprises at least one coil. For applications in which system 20 comprises external driver 34, the external driver is configured to transmit power, typically using RF energy, to stimulator via a driver wireless coupling element and the stimulator wireless coupling element, for powering stimulator 30, and to transmit and/or receive data to/from the stimulator via the wireless coupling element. Driver 34 is typically configured to be worn by the subject, such as by being coupled to a headset (such as a cellular phone headset) or a necklace, or coupled to an ear of the subject. Driver 34 typically uses the driver wireless coupling element, or a separate wireless coupling element, to wirelessly relay data to and receive data from external control unit 32. For example, such data may be transmitted using the Bluetooth protocol or another wireless communication protocol, or using an infrared signal. Alternatively, driver 34 is coupled to external control unit 32 by one or more wires.

For other applications in which system 20 does not comprise external driver 34, the functionality and components of driver 34 are incorporated into external control 34 unit, which transmits power to and sends and/or receives data to/from circuitry 40. In these applications, external control unit 32 is typically configured to be worn by the subject, such as by being coupled to a headset (such as a cellular phone headset) or a necklace, or coupled to an ear of the subject.

Whether transmitted by the external driver or directly by the control unit, such data typically includes stimulation control signals, parameters, and/or feedback information. Such data is typically transmitted only periodically, rather than constantly during stimulation. Circuitry 40 of stimulator 30 is configured to generate the stimulation waveform applied by electrodes 38, based on the configured parameters. For some applications, driver 34 or external control unit 32 combines the data and the energy into a single signal, such as by modulating the data onto the carrier frequency of the transmitted energy, in which the stimulator demodulates the received signal to obtain the data. Alternatively, the data and the energy are transmitted in separate signals.

Although electrodes 38 have been described as being applied to an SPG of the subject, for some applications the electrodes are applied to another MTS of the subject, as defined hereinabove. For some of these applications, electrodes 38 are passed through the greater palatine canal to the MTS, while for other applications the electrodes are passed through only a portion of the greater palatine canal, or are advanced to the MTS by another route.

For some applications, system 20 applies excitatory electrical stimulation to the SPG or another MTS using the following parameters:
amplitude: 0.5-10 mA
frequency: 10-30 Hz
pulse width: 100-500 μsec
cycles: 1-10 cycles per hour
cycle on/off time: 60 seconds/12 seconds, 4 seconds/15 seconds, or 30 seconds/60 seconds

For some applications, system 20 provides stimulation by applying a plurality of cycles of stimulation, each cycle including an “on” period (e.g., between 2 and 90 seconds) followed by an “off” period (e.g., between 8 and 90 seconds). Such cycles are applied a certain number of times per hour, typically spaced evenly throughout the hour. For example, if the cycles are applied four times per hour, the four cycles may be applied at the beginning of the hour, 15 minutes into the hour, 30 minutes into the hour, and 45 minutes into the hour, respectively. For some applications, each stimulation is applied in sets of two or more cycles. For example, if the stimulation is applied four times per hour, a set of two cycles may be applied at the beginning of the hour, 15 minutes into the hour, 30 minutes into the hour, and 45 minutes into the hour, respectively.

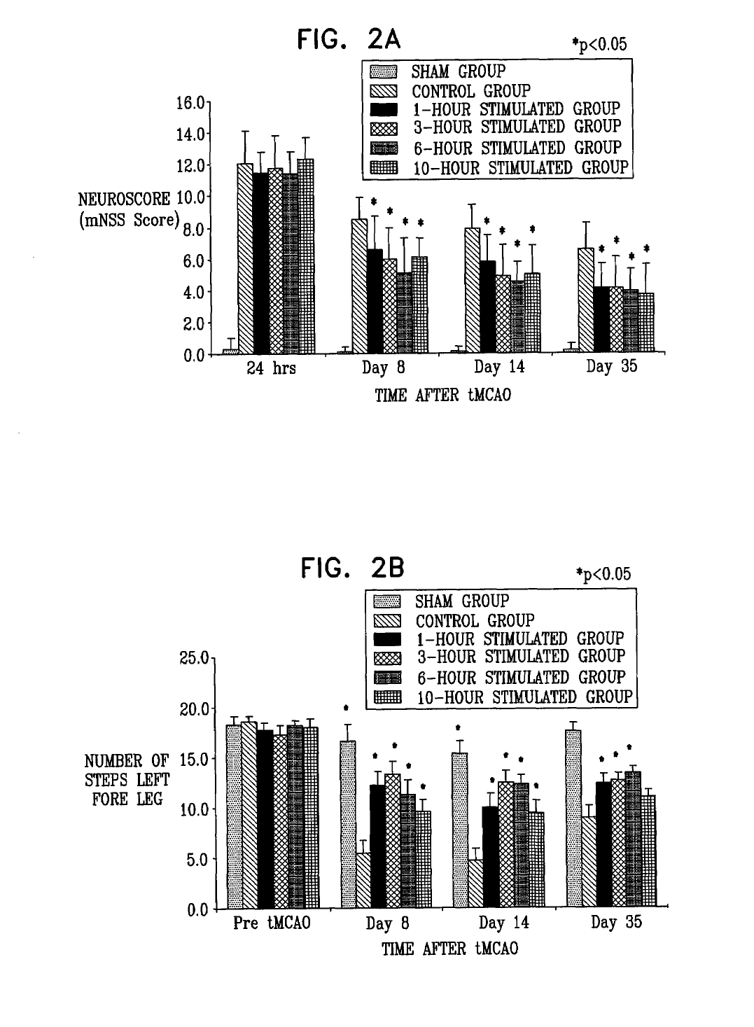

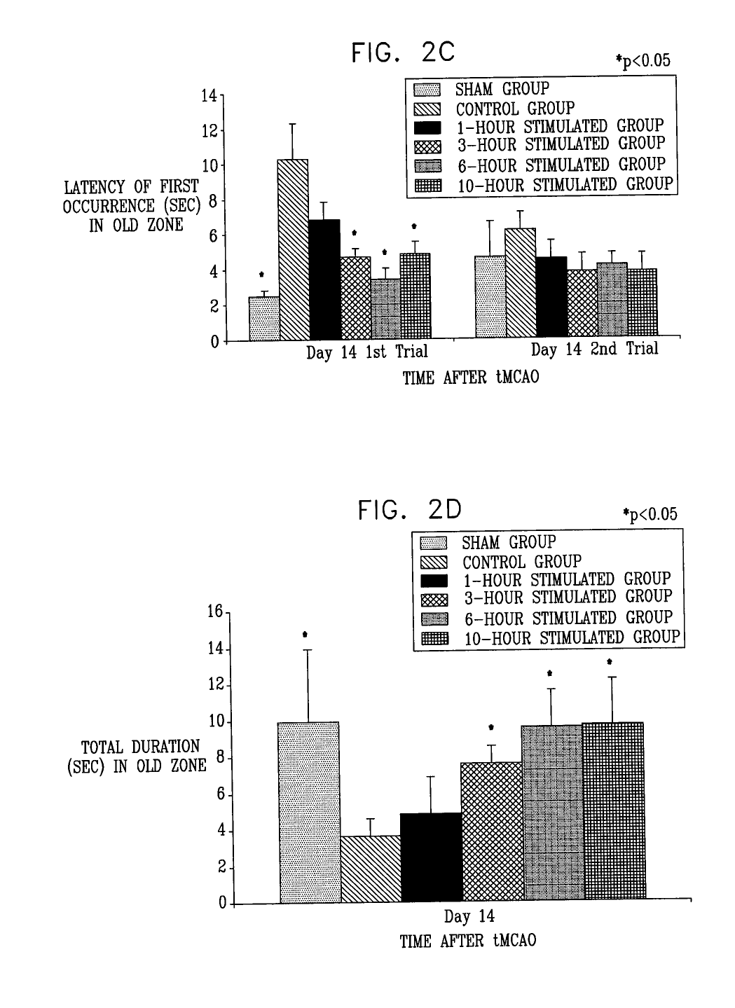

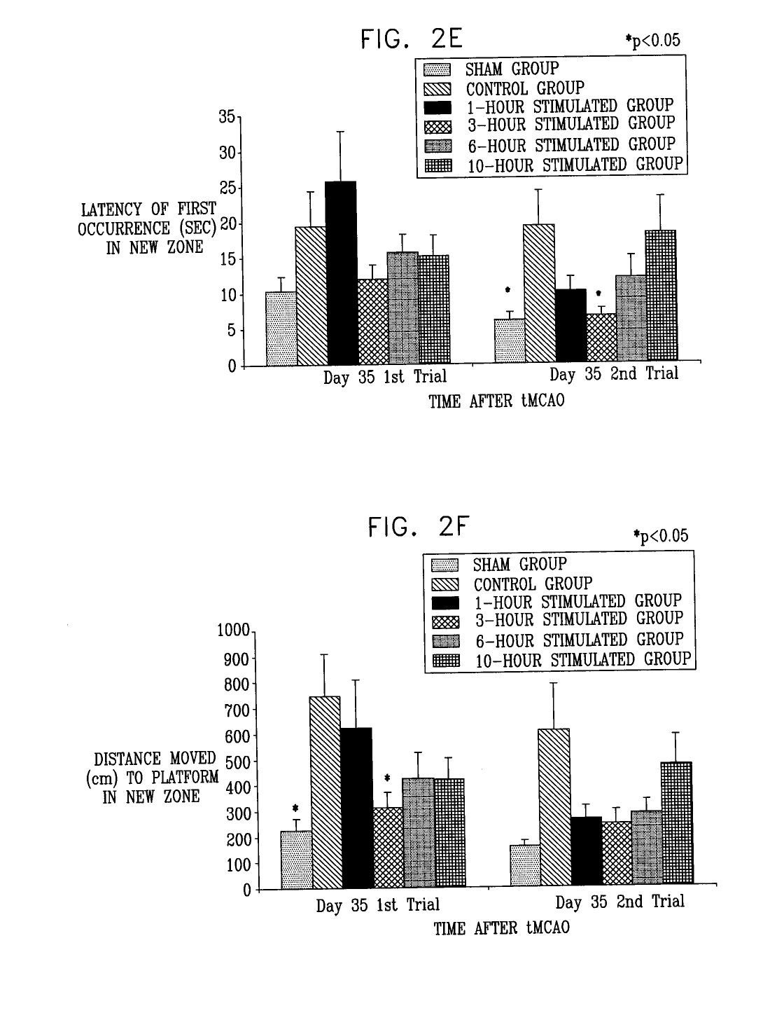

Alternatively or additionally, system 20 is configured with parameters used in the experiments described hereinbelow with reference to Tables 1 and 2 and FIGS. 2-8B.

As appropriate, placement of stimulator 30 may be facilitated by fluoroscopy, x-ray guidance, fine endoscopic surgery (FES) techniques or by any other effective guidance method known in the art, or by combinations of the aforementioned. Typically, skin temperature and/or cerebral blood flow (CBF) is measured concurrently with insertion. CBF may be measured with, for example, a laser Doppler unit positioned at the patient's forehead or transcranial Doppler measurements. Verification of proper implantation of the electrodes onto the appropriate neural structure may be performed by activating the device, and generally simultaneously monitoring CBF. For some applications, stimulator 30 is implanted using techniques described in U.S. patent application Ser. No. 10/535,024, filed Dec. 27, 2005, entitled, “Surgical tools and techniques for stimulation,” which is assigned to the assignee of the present application and is incorporated herein by reference, and/or in the above-mentioned PCT Publication WO 04/043218. For some applications, techniques described herein are performed in combination with apparatus and/or methods that are described in above-mentioned U.S. patent application Ser. No. 11/349,020.

### Augmentation of Cell Genesis

In an embodiment of the present invention, system 20 is used to apply excitatory electrical stimulation to the SPG or another MTS to augment genesis of one or more cell populations in at least one brain region of a subject suffering from an adverse cerebral condition, such as cerebral infarction, in order to improve recovery from the condition. The inventors hypothesize that such stimulation may augment the neurogenesis by increasing blood perfusion to the damaged tissue, which improves the supply of oxygen and other nutrients to the tissue, and/or by causing the release of neurotransmitters and/or neuromodulators from the SPG nerve fibers.

A method for applying such stimulation comprises identifying an electrical stimulation protocol as being suitable for augmenting genesis of one or more cell populations in the at least one brain region of the subject, and augmenting the cell genesis by applying the identified stimulation protocol to the SPG or another MTS. For some applications, the stimulation protocol is identified as being suitable for augmenting neurogenesis in the at least one brain region. Alternatively or additionally, the stimulation protocol is identified as being suitable for augmenting angiogenesis in the at least one brain region. Further alternatively or additionally, the stimulation protocol is identified as being suitable for augmenting glia-genesis in the at least one brain region. Typically, the method comprises identifying that the subject may benefit from the augmented cell genesis, and augmenting the cell genesis comprises augmenting the cell genesis responsively to identifying that the subject may benefit from the augmented cell genesis.

In the present patent application, including in the claims, augmenting cell genesis means causing an increase in a level of cell genesis compared to the cell genesis that would naturally occur if the stimulation were not applied. As mentioned above, Sun Y et al. report that ischemia itself stimulates neurogenesis. The techniques of some embodiments of the present invention cause a greater level of neurogenesis than the ischemia would stimulate in the absence of the electrical stimulation of the SPG or another MTS.

In an embodiment of the present invention, the method comprises identifying that the subject suffers from an adverse cerebral condition, and the cell genesis is augmented responsively to identifying that the subject suffers from the adverse cerebral condition. The cerebral condition causes a brain area of the subject to be diseased. For some applications, the stimulation causes cell genesis in a vicinity of the diseased brain area. Alternatively or additionally, the stimulation causes cell genesis in a brain area other than the vicinity of the diseased brain area. For some applications, the adverse cerebral condition is a cerebrovascular infarction (stroke), and the augmentation of cell genesis leads to a better prognosis for acute stroke patients. Alternatively, such stimulation augments neurogenesis in a brain suffering from another adverse cerebral condition, such as chronic cerebral hypoperfusion states (such as occur in vascular dementia and Alzheimer's disease), neurodegenerative disorders (such as Parkinson's disease) and electrical hyper-activity states (such as epilepsy).

In an embodiment of the present invention, application of the stimulation commences at least 18 hours after an occurrence of a cerebrovascular infarction. Experimental evidence described hereinbelow with reference to Table 1 supports the efficacy of stimulation commencing 24 hours after stroke.

In some embodiments of the present invention, a method is provided that comprises identifying that a subject may benefit from augmented neurogenesis, and, responsively to the identifying, applying stimulation to the SPG or another MTS of the subject. Alternatively or additionally, the method comprises identifying a stimulation protocol as suitable for augmenting neurogenesis, and applying the identified protocol to a subject who may benefit from augmented neurogenesis.

In some embodiments of the present invention, a method is provided for augmenting neurogenesis in a subject who has not been diagnosed with any neurological condition associated with reduced neurogenesis or an elevated level of brain cell death. The method comprises identifying that the subject may benefit from augmented neurogenesis, and, responsively to identifying, applying chronic, long-term stimulation to an MTS, such as the SPG. The chronic stimulation has a duration of at least two weeks, at least four weeks, at least three months, at least six months, or the remaining life of the subject. During this chronic treatment, stimulation is typically applied intermittently, such as during one session per day, or less frequently, such as depending on the severity of assessed risk. Alternatively, the stimulation is applied generally constantly, typically at a low strength.

For some applications, identifying that the subject may benefit from augmented neurogenesis comprises identifying that the subject is at least a threshold age. For example, the threshold age may be between about 50 and about 80 years. Alternatively or additionally, the subject may identified because the subject suffers from one or more symptoms of a vascular disorder, such as hypertension, peripheral vascular disease, or coronary artery disease. Alternatively or additionally, the method comprises identifying that the subject suffers from reduced cardiac output, e.g., caused by heart failure. Further alternatively or additionally, the subject may be identified as potentially benefiting from augmented neurogenesis if the subject has a family history of any type of dementia, and/or the subject has radiological findings suggestive of cerebral vascular disease or Alzheimer's disease.

### Neurogenesis Experimental Results

The inventors conducted an experiment to evaluate the level of augmentation of neurogenesis in infarcted rat brains caused by SPG stimulation. The experiment used a rat middle cerebral artery occlusion (MCAO) model of stroke. Stroke was induced in two rats, one of which served as a control, and the other of which rat was treated with SPG stimulation beginning 24 hours after stroke. The rats were sacrificed, and histopathological examinations and immunohistochemical analysis for markers of newly born cells were performed.

The following table shows cell counts in tissue taken from the proliferating zone of the brain (where new cells are generated in the brain):

TABLE 1

| | Number of DCX- | Ki-67- | Cells both DCX-positive |
|---|---|---|---|
| | positive cells | positive cells | and Ki-67-positive |
| | (% total cells) | (% total cells) | (% total cells) |
| Control | 128.3 ± 49.3 | 17.25 ± 12.23 | 8.75 ± 2.29 |
| rat | (55.1 ± 17.1) | (9.3 ± 3.8) | (4.5 ± 1.3) |
| Stimulated | 56.7 ± 27.7 | 32.33 ± 3.38 | 16.6 ± 2.3 |
| rat | (47.7 ± 4.3) | (41.0 ± 14.9) | (19.5 ± 7.2) |

(The ranges of values represent data measured in several slices taken from each rat brain.)

The doublecortin (DCX) protein is a marker for immature neurons, and is thus a marker for neurogenesis. The Ki-67 protein is a cellular marker for proliferation.

As can be seen in Table 1, a substantially greater percentage of the cells in the proliferating zone are newly born cells in the stimulated rat than in the control rat (41% vs. 9.3%), as indicated by the Ki-67 marker. Furthermore, a greater percentage of the newly born cells were neurons (positive for both the DCX and Ki-67 markers) in the stimulated rat and control rat (19.5% vs. 4.5%).

The examinations indicated that the SPG-stimulated rat had substantially more newly born cells than the control rat, and that a substantially greater percentage of the newly born cells were neuroblasts (the progenitor cells of neurons) in the SPG-stimulated rat than in the control rat.

Reference is made to FIGS. 2A-H, which are graphs showing results of an in vivo experiment assessing the effect of long-term rehabilitative SPG stimulation, measured in accordance with an embodiment of the present invention. A rat tMCAO model of stroke was used to evaluate the benefits, including neuromuscular, motility, cognitive, somatosensory, somatomotor, infarct volume benefits, of rehabilitative SPG stimulation using the techniques described herein. The stimulation was applied for seven consecutive days beginning at 24 hours after reperfusion in the tMCAO model.

94 male Sprague Dawley (SD) rats were divided into six groups, as shown in Table 2:

TABLE 2

| | | Hours of |
|---|---|---|
| | | stimulation |
| Group | No. of rats | per day |
| 1 - Control | 18 | N/A |
| 2 - Sham | 10 | N/A |
| 3 | 17 | 1 |
| 4 | 16 | 3 |
| 5 | 17 | 6 |
| 6 | 16 | 10 |

Prior to performance of any surgical procedure on the rats, the rats were trained using a series of behavior tests. Five parameter categories were evaluated using one or more tests, as follows:
Neuromuscular function—rotarod motor test, mNSS test, beam walking and balance test, stepping test, and staircase skilled reaching test;
Motility—open field test;
Learning memory (cognitive) water maze test;
Somatosensory sensation—adhesive removal test; and
Somatomotor sensation—corner turn test.

Transient MCAO (tMCAO) was performed on the right hemisphere of all of rats except those of the sham group, using the techniques described hereinabove with reference to FIGS. 11A-C. Three hours after the occlusion, reperfusion was allowed in all groups. On the day of tMCAO, the rats were anesthetized, and a bipolar electrode was implanted in contact with the SPG ipsilateral to the pMCAO (i.e., the right SPG), and connected to a controller. At 24 hours post-tMCAO (just prior to stimulation), the rats were subjected to neuroscoring using the mNSS scale, which has a score range of 0-18, where 0 represents normal and 18 represents maximum neurological defect. Rats scoring less than or equal to 9 were excluded from the experiment.

SPG stimulation was applied for seven consecutive days beginning at 24 hours post-tMCAO, using the following regime: a duty cycle of 60 seconds on/12 seconds off, with two cycles every 15 minutes, at 2 mA and 10 Hz, with a 500 μsec pulse width. The stimulation was applied for fifteen minutes every 60 minutes. The number of hours of stimulation per day was as shown in Table 2 above.

In order to assess rehabilitation, on days 8, 14, and 35 post-tMCAO, (with limited exceptions for specific tests), the rats were subjected to the same pre-procedure behavior tests used in the training, as described hereinabove. One day after the last behavior testing, the rats were sacrificed and perfused. Their brains were harvested, infarct volume was measured, and neurons were counted.

The results of the experiment included the following:
Mortality in the SPG-stimulated groups was lower than in the non-stimulated control group.
SPG stimulation generally improved neuromuscular functions (rotarod, mNSS, beam walk and balance, stepping and staircase tests) in comparison to the non-stimulated control group.
SPG stimulation improved cognitive capabilities (water maze test) in comparison to the non-stimulated control group.
There was a trend towards increased motility (open field test) in the SPG-stimulated groups.
Somatosensory sensations were enhanced in the SPG-stimulated groups in comparison to the non-stimulated control group.
Somatomotor competence was superior in the SPG-stimulated groups than in the non-stimulated control groups.
SPG stimulation resulted in higher neurons counts in cortical layer V of the ipsilateral stimulated side in comparison to the non-stimulated control group.

In summary, in the present experiment, SPG stimulation initiated 24 hours after tMCAO had advantageous results for all five parameter groups evaluated. In addition, SPG stimulation increased the number of neurons in all regions counted.

FIG. 2A is a graph showing neuroscores (mNSS test) of all six groups, measured at 24 hours, 8 days, 14 days, and 35 days after tMCAO, measured in accordance with an embodiment of the present invention. As can be seen in the graph, mNSS scores of the SPG-stimulated rats decreased in a time-dependent manner post-tMCAO, indicating the occurrence of an active restorative, rehabilitative process. SPG stimulation markedly and significantly (p<0.05) improved neurological function measured at days 8, 14, and 35 in all SPG-stimulated groups.

FIG. 2B is a graph showing the results of the stepping test performed on the left foreleg in all six groups, measured pre-tMCAO and at 8 days, 14 days, and 35 days after tMCAO, measured in accordance with an embodiment of the present invention. As can be seen in the graph, there was a significant (p<0.05) increase in left (impaired) foreleg stepping in all SPG-stimulated rats in comparison to the non-stimulated control group (with the exception of the 10-hour stimulated group at day 35). Maximum improvement was evident in the 3- and 6-hour stimulation groups at days 14 and 35, respectively.

FIGS. 2C-F are graphs showing the results of the Morris water maze (WM) task, measured in accordance with an embodiment of the present invention. The Morris WM task is a standard test of learning in which the animal repeatedly searches for a rest platform hidden beneath the surface in a pool. The test is especially sensitive to hippocampal and cortical damage, and reflects attention, memory, and learning strategy. The Morris WM task was performed on days 14 and 35 following tMCAO.

FIG. 2C is a graph showing the latency to the first occurrence in the Old Zone (as described below) in first and second trials at 14 days after tMCAO, measured in accordance with an embodiment of the present invention.

This parameter assesses the rats' functional memory. The rest platform was moved from the Old Zone (its position during training) to the New Zone (its position during testing), and the rats were expected to seek the Old Zone. The first trial showed that the SPG-stimulated rats (3-, 6-, and 10-hour stimulation) returned to the Old Zone significantly (p<0.05) more quickly than the non-stimulated rats in the control group. The second trial showed, although non-significantly, that the SPG-stimulated rats returned to the Old Zone faster than the non-stimulated controls, even though introduced to the New Zone rest platform in the first trial. The second trial thus confirmed that the SPG-stimulated rats showed enhanced remnants of functional memory.

FIG. 2D is a graph showing time spent in the Old Zone at day 14 after tMCAO, measured in accordance with an embodiment of the present invention. This parameter also assesses the rats' functional memory. As can be seen in the graph, the 3-, 6-, and 10-hour SPG-stimulated groups spent significantly (p<0.05) more time seeking the rest platform in the Old Zone in comparison to the non-stimulated control group.

FIG. 2E is a graph showing the latency to the first occurrence in the New Zone in first and second trials at day 35 after tMCAO, measured in accordance with an embodiment of the present invention. This parameter also assessed the rats' functional memory. In the first trial, the 3-, 6-, and 10-hour SPG-stimulated groups demonstrated superior, although non-significant, results in finding the New Zone, compared with the non-stimulated control group. In the second trial, all of the SPG-stimulated groups achieved better results than the non-stimulated control group. These results were significant (p<0.05) only in the 3-hour stimulated group.

FIG. 2F is a graph showing the distance moved to find the rest platform in the New Zone in first and second trials at day 35 after tMCAO, measured in accordance with an embodiment of the present invention. This parameter assessed the rats' long-term learning capability. In both trials the SPG-stimulated rats demonstrated better performance than the control group. These results were significant (p<0.05) only in the 3-hour stimulated group during the first trial.

The staircase test (results not shown) was performed to assess the rehabilitation of foreleg fine motorics. At day 14 after tMCAO the SPG-stimulated groups demonstrated better performance in the left impaired foreleg than the control group (1-, 3-, and 6-hour stimulation, significant (p<0.05) in the 3- and 6-hour stimulated rats only). At day 35 after tMCAO the SPG-stimulated groups demonstrated better performance in the left impaired foreleg, significant (p<0.05) in the 3-hour stimulated rats only.

The rotarod test (results not shown) was performed to assess the rats' ability to remain on a rotating rod. It requires a high degree of sensorimotor coordination and is sensitive to damage in the basal ganglia and the cerebellum. The only significant (p<0.05) results were in the 3-hour stimulated rats on the 35 day assessment, which remained on the rotarod significantly longer than the control group.

FIG. 2G is a graph showing the time required for the rats to remove an adhesive patch from the left foreleg, measured in accordance with an embodiment of the present invention. This test assessed both cutaneous sensitivity and sensor motor integration, and is analogous to human neurological tests used clinically in stroke patients. In the left impaired foreleg, the SPG-stimulated rats showed better results than the non-stimulated controls at all assessment days (8, 14, and 35 days). These results were significant (p<0.05) at all three assessment days in the 3- and 6-hour stimulated groups only.

The corner test (results not shown) was performed to evaluate the rats' tendency to favor a turn in the direction of the ipsilateral side of the tMCAO (i.e., the right side in the experiment). On all three assessment days (8, 14, and 35 days), all SPG-stimulated groups showed a decrease in right side turns in comparison to the non-stimulated control group. This decrease was significant (p<0.05) only on day 35 in the 1- and 6-hour stimulated rats.

The beam walk test (results not shown) was performed to evaluate sensor motor integration, specifically hind limb function. In general, all SPG-stimulated groups showed improved results in comparison to the non-stimulated control group. These results were significant (p<0.05) only on day 35 only in the 3-hour stimulated group.

The beam balance test (results not shown) was performed to assess gross vestibulomotor function, by requiring the rats to balance steadily on a narrow beam. This test is sensitive to motor cortical insults. On all assessment days (days 8, 14, and 35), all of the SPG-stimulated groups (except the 1-hour stimulated group on day 8) performed better than the non-stimulated control group. These results were significant (p<0.05) only on day 14 in the 3-hour stimulated group.

The open field test (results not shown) was performed to assess the following four parameters indicative of hippocampal and basal ganglia damage, as well as hind limb dysfunction:
Total distance moved, which decreases in cerebrally-insulted animals. All of the SPG-stimulated groups achieved enhanced movement compared to the control group on day 14 after tMCAO. These results were significant (p<0.05) only in the 3- and 6-hour stimulated groups.
Velocity, which is diminished in cerebrally-insulted animals. All of the SPG-stimulated groups achieved enhanced velocity compared to the control group on day 14 after tMCAO. These results were significant (p<0.05) only in the 3- and 6-hour stimulated groups.
Latency of first occurrence in center zone. All of SPG-stimulated groups (except the 10-hour stimulated group on day 14) showed quicker entry into the center zone in comparison to the non-stimulated control group. These results were significant (p<0.05) only on day 35 in the 1- and 3-hour stimulated groups.
Total distance moved in center zone. On day 14, the 3- and 10-hour stimulated groups achieved significantly (p<0.05) greater distance moved than the control group.

FIG. 2H is a graph showing the number of neurons in cortical layer V and II-III, measured in accordance with an embodiment of the present invention. Neuron counting was performed in cortical layers V and II-III in the non-stimulated control group and in the 3- and 6-hour SPG-stimulated groups. The number of neurons in cortical layer V was significantly (p<0.05) greater in both of these SPG-stimulated groups compared to the non-stimulated group. In cortical layers II-III there was no significant difference between the stimulated and non-stimulated groups.

There were no significant differences in body weigh between the SPG-stimulated groups and the non-stimulated control group.

### Metabolic Improvement

In an embodiment of the present invention, system 20 is used to apply excitatory electrical stimulation to the SPG or another MTS to a subject who suffers from an adverse cerebral condition, in order to improve a metabolic state of a brain area affected by the adverse cerebral condition, thereby improving recovery from the condition. For some applications, the adverse cerebral condition is a cerebrovascular infarction (stroke), and the stimulation augments the recovery of the metabolic state of the brain area in the infarction or a vicinity of the infarction. The inventors hypothesize that such stimulation may improve the metabolic state by increasing blood perfusion to the damaged tissue, which improves the supply of oxygen and other nutrients to the tissue, and/or increases washout of toxic waste products from the damaged tissue. For example, lactate is a toxic waste product which, in high concentrations, leads to acidosis of the tissue.

A method for applying such stimulation comprises identifying an electrical stimulation protocol as being suitable for improving a metabolic state of a brain area of a subject, and improving the metabolic state by applying the stimulation protocol to the SPG or another MTS. Typically, the method comprises identifying that the subject suffers from an adverse cerebral condition, and the metabolic state is improved responsively to identifying that the subject suffers from the adverse cerebral condition. For some applications, the method comprises identifying that the subject may benefit from the improved metabolic state, and the metabolic state is improved responsively to identifying that the subject may benefit from the improved metabolic state.

In an embodiment of the present invention, application of the stimulation commences at least 18 hours after an occurrence of the infarction. Experimental results obtained by the inventors support the efficacy of stimulation commencing 24 hours after stroke, as described hereinbelow with reference to FIGS. 3-8B.

For some applications, the brain area is an ischemic core of the infarction. The stimulation improves metabolism even the core, in which cells have a high likelihood of having died during the infarction. For some applications, identifying the stimulation protocol comprises identifying the stimulation protocol as suitable for reviving at least a portion of the ischemic core. Alternatively or additionally, the brain area is the periphery around the ischemic core.

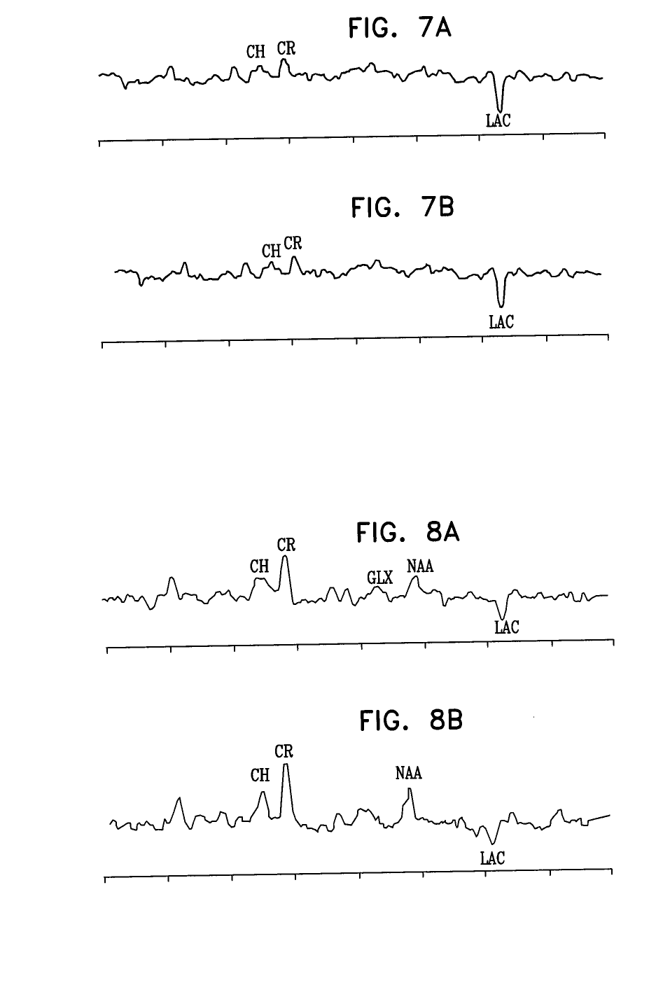

In an embodiment of the present invention, electrical stimulation of the SPG or another MTS reduces lactate levels in the acute phase of a cerebrovascular infarction, or during another adverse cerebral condition. Such a reduction in lactate levels results in a better metabolic state for the surviving cells. As described hereinbelow with reference to FIGS. 7A-B and 8A-B, preliminary experimental results in a rat model indicate the SPG stimulation reduces such lactate levels in the acute phase of the infarction. For some applications, identifying the stimulation protocol in the stimulation method comprises identifying the stimulation protocol as suitable for enhancing clearance of lactate from the brain area.

In an embodiment of the present invention, stimulation of the SPG or another MTS augments the recovery of the metabolic state of an infarcted brain area, which leads to a better prognosis for acute stroke patients. Alternatively, such stimulation augments the recovery of the metabolic state of a brain area affected by another adverse cerebral condition, such as chronic cerebral hypoperfusion states (such as occur in vascular dementia and Alzheimer's disease), neurodegenerative disorders (such as Parkinson's disease), and electrical hyperactivity states (such as epilepsy where there is a higher metabolic demand in the affected brain areas).

In an embodiment of the present invention, a method is provided for improving a metabolic state of the brain in a subject who has not been diagnosed with any neurological condition associated with a reduced level of brain metabolism. The method comprises identifying that the subject may benefit from an improved metabolic state, and, responsively to identifying, applying chronic, long-term stimulation to an MTS, such as the SPG. The chronic stimulation has a duration of at least two weeks, at least four weeks, at least three months, at least six months, or the remaining life of the subject. During this chronic treatment, stimulation is typically applied intermittently, such as during one session per day, or less frequently, such as depending on the severity of assessed risk. Alternatively, the stimulation is applied generally constantly, typically at a low strength.

### Metabolic State Experimental Results

Reference is made to FIGS. 3-6, which are bar graphs showing experimental results obtained during a rat experiment performed in accordance with an embodiment of the present invention. The inventors conducted this experiment to assess the ability of SPG stimulation to augment stroke recovery in MCAO rats.

Transient middle cerebral artery occlusion (t-MCAO) was induced in twenty Wistar rats. 7 of the rats served as a control group, and the remaining 6 rats were treated with SPG stimulation beginning 18±2 hours post-MCAO. Longitudinal ¹H magnetic resonance spectroscopic imaging (¹HMRSI) and diffusion MRI (DWI) were used to evaluate ischemic brain condition of the stimulated and control rats at 16±2 h, 8 days, and 28 days post-MCAO. In addition, the two groups were evaluated by modified neurological severity score (mNSS). N-Acetyl-Aspartate (NAA) levels, as obtained from ¹HMRSI, and a damage index, computed from ADC maps, were used to determine the pathophysiological state of the control and SPG-treated groups.

As described in detail below, the inventors found that levels of NAA, which is considered to be a marker for neuronal density and viability levels, in the stimulated and control rats were the same 16±2 hours post-MCAO (0.52±0.03, 0.54±0.03). 28 days post-MCAO, NAA levels were significantly higher in the stimulated group (0.60±0.04) compared to the control group (0.50±0.04) (P<0.05). This effect was more pronounced for regions with low initial NAA values; in these regions, NAA increased from 0.16±0.03 to 0.32±0.03 in the stimulated group (P=0.04), and from 0.16±0.03 to 0.10±0.03 (P=0.20) in the control group. The inventors believe that regions with the lowest initial NAA values generally correspond to the ischemic core of the infarction, and that regions with intermediary initial NAA values generally correspond to the ischemic penumbra. In addition, the damage index, calculated based on DWI, showed significant deterioration for the controls which was not observed for the stimulated animals.

NAA recovery after cerebral ischemia has been discussed previously in the literature (see, for example, the above-mentioned articles by Weber R et al. and Sager T N et al.). In the above-mentioned article by Moffett J R et al., the following possible contributions for this observation were suggested: (i) surviving neuronal cells in the infarction area renew their ability to synthesize NAA, (ii) different cells begin to express NAA, (iii) production of NAA from N-Acetylaspartylglutamate (NAAG) by glial cells present in the infarction core, and (iv) neurogenesis following the ischemic event. Based on the experimental results described herein, the inventors hypothesize that the increase in the NAA levels in the stimulated group compared to the control group is due to renewal of the NAA synthesis by surviving neuronal cells. Because electrical stimulation of the SPG increases CBF in the infarction region, and NAA synthesis is dependent on the energy level in cells, such synthesis is reduced immediately after the ischemic event and the depletion of energy storage. Therefore, the increase in NAA levels in the damaged area may be caused by the increase in the energy supply to these areas, as a result of the increase in CBF caused by electrical stimulation of the SPG. In contrast, as reported in the above-mentioned article by Franke C et al., no NAA recovery had been found after tPA treatment.

The twenty Wistar rats were male, and weighed 290±10 g. The rats were anesthetized with isoflurane (4% for induction, 2% for surgery) and ventilated with N₂O:O₂ (70:30%) mixture. 24 hours prior to the MCAO procedure, the head skin of the rat was clipped and cut along the midline (cranio-caudal axis). The skin and the orbital structures of the right side (ipsilateral to subsequent t-MCAO) were retracted laterally to expose the ethmoidal foramen and the ethmoidal nerve (i.e., the postganglionic parasympathetic nerve fibers from the SPG). A hook stimulating electrode, which was subsequently used to generate the electrical pulses, was hooked onto the exposed fibers. The wire of the electrode was glued onto the skull, and the receiver was placed under the skin on the nape of the animal. The surgical wound was closed above the right orbit. Rats of both the stimulated group and the control group were implanted.

Prior to the t-MCAO procedure, the rats were again anesthetized with isoflurane (4% for induction, 2% for surgery) and ventilated with N₂O:O₂ (70:30%) mixture. t-MCAO was induced by intraluminal suture occlusion of the right MCA, using the suture model as described by Spratt N J et al., “Modification of the method of thread manufacture improves stroke induction rate and reduces mortality after thread-occlusion of the middle cerebral artery in young or aged rats,” J Neurosci Methods 2006; 155:285-290, which is incorporated herein by reference. In brief, 4-0 monofilament nylon suture (SMI, Belgium) was coated with silicon (Wacker-Chemie, Germany) and inserted through the proximal external carotid artery into the internal carotid artery and then into the circle of Willis, effectively occluding the MCA. The suture was placed for two hours and subsequently removed. The surgical wound was closed and the animals returned to their cages for a recovery period of approximately sixteen hours.

Arterial blood samples were taken before, and immediately after the MCAO, to measure pH, PaO₂ and PaCO₂. Body temperature was maintained at 37.0±0.5° C., using an electrical heating pad. Five rats died during the first sixteen hours, prior to the MR experiment. Six rats were stimulated. Two of nine rats in the control group died within the first week.

Magnetic resonance imaging (MRI) and spectroscopy (MRS) are widely used for investigating, in vivo, neurological disorders in general, and ischemic stroke in particular. The versatility of the MR technique enables non-invasive studying of not only the progression of neurological pathology during longitudinal follow-up, but also the evaluation of the pathophysiological state of the studied tissue, without the need of animal sacrificing. Of the available MR approaches, diffusion weighted imaging (DWI) is a powerful tool for early stroke detection. DWI enables observation of ischemic tissues within minutes following the ischemic event. Apparent diffusion coefficient (ADC) maps, calculated from DWI, may be used to evaluate infarction size as well as tissue condition, and correlate to infarction sizes evaluated from histology. The acute stage of the ischemic stroke is characterized, inter alia, by cellular swelling (cytotoxic edema) and increase in the extracellular tortuosity that reduces the ADC of the water molecules in the ischemic region. In the chronic stage of ischemic stroke, high ADC values (compared to the contralateral regions) are observed for the infarction area as a result of the necrotic process that includes cells death and membrane loss. Therefore, ADC values computed at the chronic stage and normalized to the contralateral hemisphere values can predict tissue condition. Simultaneously with the changes in the diffusion characteristics of water within the ischemic region, changes in levels of different brain metabolites can be detected by different MRS methodologies. Of these metabolites, N-acetyl-aspartate (NAA) is considered to be a marker for neuronal density and viability, affording information about the neuronal tissue condition and it may predict the brain status at the chronic stage (see, for example, the two above-mentioned articles by Demougeot C et al.).

The MR experiments were performed using a 7T/30 cm BioSpec system (Bruker, Germany) equipped with a BGU20 gradient system, capable of producing pulse gradients of 400 mT/m in each of the three dimensions. A transmit body coil (ID=150 mm) and a receive surface coil (ID=15 mm) actively decoupled were used to acquire MRI and MRS data. Control (n=7) and treated (n=6) rats were examined by MRS and MRI under isoflurane anesthesia (induction 4.0%, maintenance 1.5%) in N₂O:O₂ gas mixture. Each rat was examined at three time points: 16±2 h, 8 days and 28 days post-t-MCAO.

T2 weighted MRI images (T₂WI) were collected using the RARE sequence (RARE factor=8) with the following parameters: field of view (FOV) of 25.6×25.6 mm² and 256×128 digital resolution reconstructed to 256×256. Eight continuous 2 mm slices were collected, using TR/TE of 3000/75 ms with four averages in 3 minutes and 45 seconds. ADC maps were calculated from two spin-echo four-shot echo planar images (EPI), collected with and without diffusion sensitizing gradient pulses and with the following parameters: δ=4.5 ms, Δ=40 ms and G=173 mT/m, resulting in a bₘₐₓ of 1500 s/mm². The same geometry (i.e., slices and FOV) used in T₂WI was used in the DWI protocol. For diffusion images, the matrix was 96×96 reconstructed to 128×128 with TR/TE=2000/53 ms. The entire DWI protocol was completed within two minutes.

2 mm slice-selected two-dimensional (2D) ¹H-MRSI was performed with the following parameters: FOV of 25.6×25.6 mm² with VAPOR water suppression, a matrix of 8×8 reconstructed to 16×16, resulting in 256 voxels of 1.6×1.6×2.0 mm³. TR/TE=2000/135 ms were used with 32 averages. The total collection time of the ¹H-MRSI data was 47 minutes.

After the completion of the first MR protocol (16±2 hours post-t-MCAO) the SPG-stimulated rats (treated group) were moved to a dedicated RF activation cage (BrainsGate, Israel) which enables wireless stimulation. The following electrical stimulation protocol was applied: two 60-second long pulses separated by 12 seconds of off-time, applied every 15 minutes (8 pulses per hour). Each pulse was of 2 mA amplitude, 0.5 ms pulse width and 10 Hz frequency. SPG stimulation started 18±2 hours post-t-MCAO surgery and was applied for 3 hours, for seven consecutive days.

2D ¹H-MRSI raw data were split into 256 individual NMR spectra. 15 spectra from the ipsilateral hemisphere and their respective contralateral spectra were used to calculate normalized total-NAA values for each examined animal at all three time points, i.e., at 16±2 h, 8 days, 28 days post t-MCAO. NAA integration values were determined by using the line fitting procedure of MestRe-C software (Mestrelab Research, Santiago de Compostela, Spain). The NAA integration values of the ipsilateral voxels were normalized to the NAA values obtained for the contralateral voxels, to determine the normalized level of the NAA in the ischemic side compared to the non-ischemic side.

ADC maps were calculated from two contiguous EPI diffusion experiments with b-values of 1.5 and 1500 s/mm². Lesion volumes were calculated, blindly, for all slices, by manually choosing the area of DWI abnormality in each slice and multiplying it by the slice thickness. 16±2 hours post-t-MCAO, regions having lower ADC values compared to the contralateral hemisphere were analyzed. 28 days post-t-MCAO, regions with higher ADC values compared to the contralateral hemisphere values were chosen for the lesion volumes calculation.

The damage index was calculated from the ADC maps at the first and last time points. First, the normalized lesion values (NLVs) were calculated by dividing the average ADC value in the lesion area by the average ADC value of its respective contralateral region of interest (ROI):

$$N L V = \frac{AverageLesionValue}{AverageContraLateralValue} \qquad ( Equation 1 )$$

Since the ADC values of the lesion area obtained 16±2 hours after the stroke were smaller than those of the contralateral ROI, the damage index was calculated using the following equation:

DI(16 h)=(1−NLV)×LV (Equation 2)

Such a calculation enables evaluation of the injured tissue condition relative to the normal contralateral tissue. As expected, 28 days post-t-MCAO, the ADC values of the lesion area were higher compared to those observed in the contralateral ROI. In this case the damage index was calculated using the following equation:

DI(1 month)=(NLV−1)×LV (Equation 3)

Total lesion volume and total damage index were calculated by accumulating all legion volumes and damage indices, respectively, obtained for each animal 16±2 hours post t-MCAO and 28 days post-t-MCAO.

A neurological modified Neuro Severity Score (mNSS) test, scale 0-18, was performed at the three time points: (1) 16±2 hours post-t-MCAO (before the first stimulation of the treated group), (2) 8 days and (3) 28 days post occlusion.

The results were analyzed by two-tails Student's t-Test. P<0.05 was considered significant.

All rats involved in the experiment had normal physiological parameters, i.e., pH, PO₂, PCO₂ and temperature (maintained at 37.5° C. by electrical heating pad), before, during and after occlusion.

FIG. 3 shows the changes in the total normalized NAA values determined from ¹H-MRSI for the three experimental time points post-t-MCAO. As can be seen in the figure, there was a significant difference between the normalized NAA values of the two groups at the end of the study (day 28), whereas such a difference was not observed at the first time point, i.e., 16±2 hours post-t-MCAO. The stimulated and control groups started from similar averaged values of total normalized NAA values of 0.52±0.03 and 0.54±0.03, respectively (P=0.7), and reached different averaged normalized NAA values of 0.60±0.04 and 0.50±0.04, respectively, 28 days post-t-MCAO (P<0.05).

To obtain more specific information from the MRS data, the normalized NAA values in the ischemic hemisphere at 16±2 hours post t-MCAO were classified into three categories: (a) voxels with normalized NAA values greater than 0.7, (b) voxels with normalized-NAA values between 0.4 and 0.7, and (c) voxels with normalized NAA values less than 0.4.

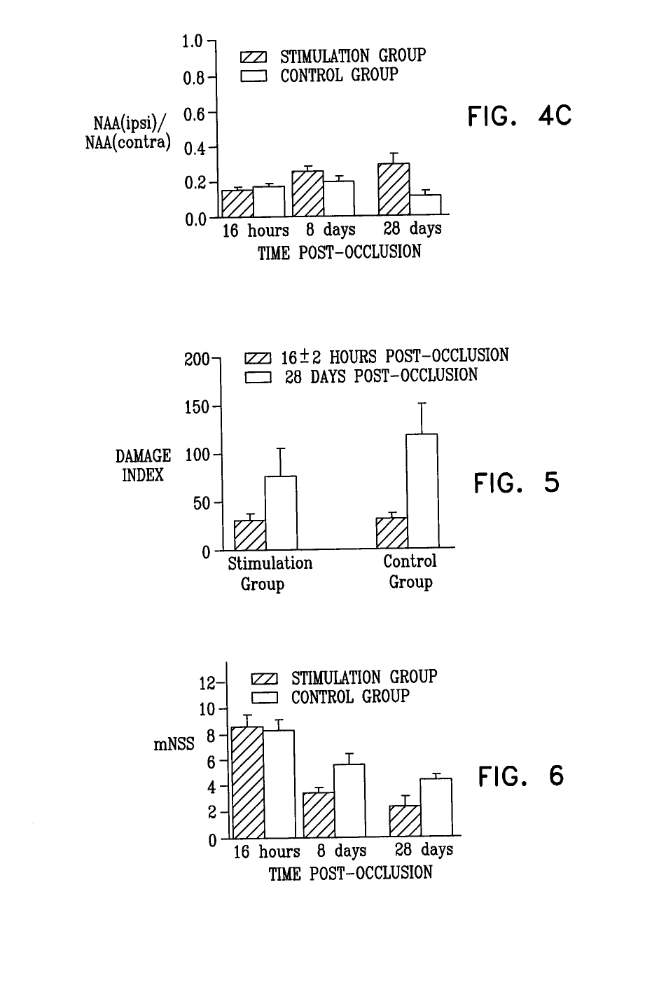

FIGS. 4A-C show summarized changes in the NAA values of these categories, respectively. FIG. 4A shows that there were no significant differences between SPG-stimulated and control groups at all three time points for voxels with normalized NAA values greater than 0.7.

FIG. 4B shows that in the voxels having initial normalized-NAA values between 0.4 and 0.7, there was an increase in normalized NAA values in the SPG-stimulated group compared to the control group, in which such an improvement in the normalized NAA was not observed. For these voxels, the normalized-NAA values of the SPG-stimulated group increased from 0.54±0.02 (16±2 hours) to 0.64±0.05 (day 8) and to 0.69±0.04 (day 28), while those of the rats in the control group did not change significantly (from 0.57±0.04 at 16±2 hours to 0.59±0.05 28 days post t-MCAO).

FIG. 4C shows that the greatest response to treatment was obtained in the voxels in which normalized NAA values were less than 0.4 at 16±2 hours post occlusion. These voxels, which showed the most dramatic reduction in NAA levels 16±2 hours post-occlusion, also exhibited the most dramatic response to treatment. For these voxels, the control group showed a decrease in the normalized NAA levels with time, from 0.16±0.03 at 16±2 hours to 0.10±0.03 28 days post-occlusion, whereas the treated group showed the opposite trend. In the SPG-stimulated animals, NAA levels from these voxels significantly improved from 0.16±0.03 at 16±2 hours to 0.32±0.03 28 days post MCAO (P=0.01). For these voxels, although the control and SPG-stimulated groups had had the same initial NAA values at 16±2 hours post occlusion (0.16±0.03, P=0.97), at 28 days post occlusion a dramatic difference in the normalized NAA values of the SPG-stimulated animals (0.32±0.07) and the controls (0.10±0.03, P=0.007) was found.

FIG. 5 shows the damage index for the two groups as computed from the diffusion MRI 16±2 hours and 28 days post t-MCAO. The damage index was not calculated for the day 8 time point, because of pseudo-normalization that may occur at this time point. As can be seen in the figure, both groups began with the same damage index values (28±18 for the SPG-stimulated animals and 28±15 for the control rats, P=0.98). The DWI data show that less deterioration in the damage index occurred in the SPG-stimulated group than in the control group. For the SPG-treated animals, the calculated damage indices did not change significantly in the day 28 follow up (P=0.15). However, the deterioration in the damage indices of the untreated animals was statistically significant (P=0.03).

FIG. 6 shows the average mNSS values obtained for the two studied groups, at all three experimental time points. Both groups began with approximately the same neuronal score, with mNSS values of 8.5±1.0 (for the SPG-stimulated group) and 8.3±0.8 (for the control group, P=0.86). Eight days post-t-MCAO, a significant difference in the mNSS of the two groups (5.6±0.8 for the control rats and 3.8±0.4 for the SPG-treated animals, P=0.04) was found. 28 days post-t-MCAO, the difference in the mNSS between the groups was maintained (4.3±0.9 versus 2.3±0.5) but somewhat less significant (P=0.08).

Reference is made to FIGS. 7A-B and 8A-B, which show magnetic resonance spectra obtained during an experiment conducted by the inventors, measured in accordance with an embodiment of the present invention. The inventors conducted this experiment to assess the ability of SPG stimulation to reduce lactate levels in MCAO rats.

Transient middle cerebral artery occlusion (t-MCAO) was induced in 4 Wistar rats, using the technique described hereinabove with reference to FIGS. 2-5. 2 of the rats served as a control group, and the remaining 2 rats were treated with SPG stimulation beginning 24 hours post-MCAO. Longitudinal ¹H magnetic resonance spectroscopic imaging (¹HMRSI) was used to evaluate lactate levels at 20 hours post-MCAO (prior to SPG stimulation) and at 27 hours (immediately after the completion of SPG stimulation in the treated group). 2 mm slice-selected ¹H-MRSI was performed using a 7T/30 cm Biospec system (Bruker, Germany) equipped with a BGU20 gradient system, capable of producing pulse gradients of 400 mT/m in each of the three dimensions, with the following parameters: FOV of 25.6×25.6 mm² with VAPOR water suppression, one voxel −3×3×3 mm (dimensions) TR/TE=3500/135 ms, NA=128, DS=8, exposure time}=8 minutes. Spectral lines were determined by taking an average over the whole voxel. ¹H-MRSI measurements were taken in each session from four different locations:
1) the ipsilateral ischemic core, as defined defined by the T2 image;
2) the same area as the ischemic core, but on the contralateral side;
3) the ipsilateral ischemic penumbra, as defined defined by the T2 image; and
4) the same area as the ischemic penumbra, but on the contralateral side.

After the completion of the first MR protocol (20 hours post-MCAO) the SPG-stimulated rats (treated group) were moved to a dedicated RF activation cage (BrainsGate, Israel) which enables wireless stimulation. The following electrical stimulation protocol was applied: two 60-second long pulses separated by 12 seconds of off-time, applied every 15 minutes (8 pulses per hour). Each pulse was of 2 mA amplitude, 0.5 ms pulse width and 10 Hz frequency. SPG stimulation started 24 hours after t-MCAO surgery and was applied for 3 hours, during a single stimulation session.

¹H-MRSI raw data respective contralateral spectra were used to calculate normalized total-LAC values for each examined animal at two time points, at 20 hours post-t-MCAO and immediately post-t-MCAO. Lactate integration values were determined by using the line fitting procedure of MestRe-C software (Mestrelab Research, Santiago de Compostela, Spain). The lactate integration values of the ipsilateral voxels were normalized to the lactate values obtained for the contralateral voxels, to determine a normalized level of lactate in the ischemic side compared to the non-ischemic side.

FIGS. 7A and 7B show magnetic resonance spectra of an infarction core before and after SPG stimulation, respectively, measured in accordance with an embodiment of the present invention. As described above, spectra were also measured for the contralateral side (not shown). The average normalized lactate levels at the ipsilateral core were 62 prior to SPG stimulation, and 54 after three hours of SPG stimulation, representing a 12.5% reduction in lactate level. This reduction is reflected by a reduction of the negative lactate peak shown in the figures.

FIGS. 8A and 8B show magnetic resonance spectra of an infarction penumbra before and after SPG stimulation, respectively, measured in accordance with an embodiment of the present invention. As described above, spectra were also measured for the contralateral side (not shown). The average normalized lactate levels at the ipsilateral penumbra were 46 prior to SPG stimulation, and 40 after three hours of SPG stimulation, representing a 15% reduction in lactate level. This reduction is reflected by a reduction of the negative lactate peak shown in the figures.

The inventors hypothesize that these observed reductions in lactate levels were caused by the partial transition from anaerobic respiration to aerobic respiration as the metabolic state of the damaged cells improved because of the applied SPG stimulation, because of a higher efflux of lactate from the ischemic tissue, or from a combination of these two mechanisms.

In an embodiment of the present invention, the stimulation described hereinabove (for augmenting cell genesis, or for improving metabolic state) is applied on a chronic, long-term basis, i.e., for at least one week, such as at least two weeks, at least four weeks, at least three months, at least six months, or longer, such as for the remainder of the subject's life. During this chronic treatment, stimulation is typically applied intermittently, such as during one session per day, or less frequently, such depending on the severity of assessed risk. For some applications, each session has a duration of between 1 minute and 6 hours, such as at least 5 minutes or at least 15 minutes, or between 2 and 4 hours, e.g., about 3 hours or about 6 hours, or more than 6 hours. Alternatively, the system is configured to apply such stimulation generally constantly, i.e., 24 hours per day. Further alternatively, the stimulation is applied less frequently than every day, such as once every other day (e.g., at least one minute during every 48 hours), or more frequently than once per day, such as during two sessions per day.

For some applications, the stimulation is applied during a plurality of stimulation periods which includes at least first and last stimulation periods. System 20 sets an inter-period interval between initiation of the first period and initiation of the last period to be at least 24 hours. For example, the first stimulation period may occur from 1:00 P.M. to 4:00 P.M. on a Monday, and the last stimulation period may occur from 1:00 P.M. to 4:00 P.M. on a Tuesday of the same week. Optionally, stimulation is applied during at least one additional stimulation period between the first and last periods. For example, stimulation may be additionally applied from 1:00 A.M. to 4:00 A.M. on the Tuesday. For some applications, the first period concludes simultaneously with the initiation of the last period, i.e., the stimulation is applied constantly from the beginning of the first period until the conclusion of the last period. For example, the stimulation may be applied constantly from 1:00 P.M. on Monday, January 1 to 4:00 P.M. on Tuesday, January 2, or constantly from 1:00 P.M. on Monday, January 1 to 4:00 P.M. on Monday, January 29. Alternatively, the initiation of the last stimulation period occurs after a conclusion of the first stimulation period, such that the stimulation is not applied during at least one non-stimulation period between the conclusion of the first stimulation period and the initiation of the last stimulation period.

For some applications, the system sets the inter-period interval to be at least 48 hours, such as at least one week, at least two weeks, or at least four weeks. When using such greater inter-period intervals, the system typically, but not necessarily, applies stimulation during at least several additional stimulation periods between the first and last stimulation periods. For some applications, such additional stimulation periods may include a plurality of daily stimulation periods, applied on every day between the initiation of the first stimulation period and the initiation of the last stimulation period. For example, the first stimulation period may occur from 1:00 P.M. to 4:00 P.M. on Monday, January 1, the last stimulation period may occur from 1:00 P.M. to 4:00 P.M. on Monday, January 8, and the additional daily stimulation periods may occur from 1:00 P.M. to 4:00 P.M. on each day from Tuesday, January 2 through Sunday, January 7, inclusive. For some applications, stimulation is applied for at least 30 minutes every day (e.g., at least 60 minutes every day) between the initiation of the first stimulation period and the initiation of the last stimulation period. For some applications, stimulation is applied during a plurality of non-continuous stimulation periods during each 24-hour period between the initiation of the first stimulation period and the initiation of the last stimulation period. For example, the first stimulation period may occur from 1:00 P.M. to 4:00 P.M. on Monday, the last stimulation period may occur from 1:00 P.M. to 4:00 P.M. on Wednesday, and stimulation may be applied during additional stimulation periods from (a) 1:00 A.M. to 4:00 A.M. on Tuesday, (b) from 1:00 P.M. to 4:00 P.M. on Tuesday, and (c) from 1:00 A.M. to 4:00 A.M. on Wednesday, such that stimulation is applied during two stimulation periods during the 24-hour period from 1:00 P.M. on Monday to 1:00 P.M. on Tuesday, and during two stimulation periods during the 24-hour period from 1:00 P.M. on Tuesday to 1:00 P.M. on Wednesday.

For some applications, the system is configured to set the inter-period interval to be no more than a maximum value, such as three, six, nine, or twelve months. For some applications, the system comprises a user interface, which enables a healthcare worker to enter a value for the inter-period interval. The system typically rejects values that are greater than the maximum value, such as by requiring the healthcare worker to enter another value, or by using the maximum value instead of the entered value. Alternatively, the system notifies the healthcare worker if the entered value is greater than the maximum value; optionally, the system allows the healthcare worker to override the notification.

For some applications, the system is configured to store a maximum total time of stimulation per each time period having a given duration, and to apply the stimulation no more than the maximum total time per each time period having the given duration. For example, the given duration of each time period may be 24 hours. Typical values for the maximum total time of stimulation per 24-hour period include one hour, three hours, six hours, ten hours, and twelve hours. For some applications, the maximum total time of stimulation is predetermined, e.g., by the manufacturer of the system, while for other applications, a healthcare worker enters the maximum total time of stimulation into the system.

As used in the present application, including the claims, a “stimulation period” includes an entire period during which stimulation is applied, even though current is applied to the site only during a portion of the period, because of the duty cycle, on/off periods, and/or frequency of the current, for example.

For some applications, the stimulation is applied bilaterally to both SPGs, while for other applications, the stimulation is applied unilaterally to the MTS (e.g., the SPG) that supplies the more affected hemisphere of the brain. For some applications in which the stimulation is applied bilaterally, techniques are used that are described in U.S. application Ser. No. 11/573,993, filed Feb. 19, 2007, entitled, “Concurrent bilateral SPG modulation,” which is assigned to the assignee of the present application and is incorporated herein by reference.

“Strength,” as used in the present application, including the claims, means a total charge applied to an MTS in a given time period, e.g., one minute, one hour, or one day. Strength is increased or decreased by changing one or more parameters of the applied stimulation, such as the amplitude, number of cycles in a given time period, frequency, pulse width, or duty cycle (e.g., ratio of “on” to “off” time within a given cycle), as described hereinbelow in greater detail.

In an embodiment of the present invention, techniques described herein are performed in conjunction with techniques described in U.S. Pat. No. 7,117,033, which is incorporated herein by reference.

In an embodiment of the present invention, bipolar stimulation is applied, in which a first electrode is applied to a first MTS, and a second electrode is applied to a second MTS.

In some embodiments of the present invention, techniques described herein are practiced in combination with techniques described in one or more of the references cited in the Background of the Invention section hereinabove and/or in combination with techniques described in one or more of the patent applications cited hereinabove.

The scope of the present invention includes embodiments described in the following patent applications, which are assigned to the assignee of the present patent application and are incorporated herein by reference. In an embodiment of the present invention, techniques and apparatus described in one or more of the following applications are combined with techniques and apparatus described herein:
U.S. Provisional Patent Application 60/203,172, filed May 8, 2000, entitled, “Method and apparatus for stimulating the sphenopalatine ganglion to modify properties of the BBB and cerebral blood flow”
U.S. patent application Ser. No. 10/258,714, filed Oct. 25, 2002, which issued as U.S. Pat. No. 7,120,489
U.S. Provisional Patent Application 60/364,451, filed Mar. 15, 2002, entitled, “Applications of stimulating the sphenopalatine ganglion (SPG)”
U.S. Provisional Patent Application 60/368,657, filed Mar. 28, 2002, entitled, “SPG Stimulation”
U.S. Provisional Patent Application 60/376,048, filed Apr. 25, 2002, entitled, “Methods and apparatus for modifying properties of the BBB and cerebral circulation by using the neuroexcitatory and/or neuroinhibitory effects of odorants on nerves in the head”
U.S. Provisional Patent Application 60/388,931, filed Jun. 14, 2002, entitled “Methods and systems for management of Alzheimer's disease,” PCT Patent Application PCT/IL03/000508, filed Jun. 13, 2003, claiming priority therefrom, and U.S. patent application Ser. No. 10/518,322 in the national stage thereof, which published as US Patent Application Publication 2006/0020299
U.S. Provisional Patent Application 60/400,167, filed Jul. 31, 2002, entitled, “Delivering compounds to the brain by modifying properties of the BBB and cerebral circulation”
U.S. Provisional Patent Application 60/426,180, filed Nov. 14, 2002, entitled, “Surgical tools and techniques for sphenopalatine ganglion stimulation,”
PCT Patent Application PCT/IL03/000966, filed Nov. 13, 2003, a U.S. patent application Ser. No. 10/535,024 in the national stage thereof, which published as US Patent Application Publication 2006/0195169
U.S. Provisional Patent Application 60/426,182, filed Nov. 14, 2002
PCT Patent Application PCT/IL03/000967, filed Nov. 13, 2003, entitled, “Stimulation circuitry and control of electronic medical device,” and U.S. patent application Ser. No. 10/535,028 in the national stage thereof
U.S. patent application Ser. No. 10/294,310, filed Nov. 14, 2002, which issued as U.S. Pat. No. 7,146,209
PCT Patent Application PCT/IL03/000631, filed Jul. 31, 2003, entitled, “Delivering compounds to the brain by modifying properties of the BBB and cerebral circulation,” which published as PCT Publication WO 04/010923, and U.S. patent application Ser. No. 10/522,615 in the national stage thereof
U.S. Pat. No. 6,853,858 to Shalev
U.S. patent application Ser. No. 10/783,113, filed Feb. 20, 2004, which issued as U.S. Pat. No. 7,117,033
U.S. Provisional Patent Application 60/426,181, filed Nov. 14, 2002, entitled, “Stimulation for treating ear pathologies,”
PCT Patent Application PCT/IL03/000963, filed Nov. 13, 2003, which published as PCT Publication WO 04/045242, and U.S. patent application Ser. No. 10/535,025 in the national stage thereof
U.S. Provisional Patent Application 60/448,807, filed Feb. 20, 2003, entitled, “Stimulation for treating autoimmune-related disorders of the CNS”
U.S. Provisional Patent Application 60/461,232, filed Apr. 8, 2003, entitled, “Treating abnormal conditions of the mind and body by modifying properties of the blood-brain barrier and cephalic blood flow”
PCT Patent Application PCT/IL03/00338, filed Apr. 25, 2003, and U.S. patent application Ser. No. 10/512,780 in the national stage thereof, which published as US Patent Application 2005/0266099
U.S. Provisional Patent Application 60/506,165, filed Sep. 26, 2003, entitled, “Diagnostic applications of stimulation”
U.S. patent application Ser. No. 10/678,730, filed Oct. 2, 2003, which published as US Patent Application 2005/0074506
PCT Patent Application PCT/IL04/000897, filed Sep. 26, 2004, entitled, “Stimulation for treating and diagnosing conditions,” which published as PCT Publication WO 05/030025
U.S. Provisional Patent Application 60/604,037, filed Aug. 23, 2004, entitled, “Concurrent bilateral SPG modulation”
PCT Patent Application PCT/IL05/000912, filed Aug. 23, 2005, entitled, “Concurrent bilateral SPG modulation,” which published as PCT Publication WO 06/021957, and U.S. patent application Ser. No. 11/573,993 in the national stage thereof
U.S. patent application Ser. No. 10/952,536, filed Sep. 27, 2004, entitled, “Stimulation for treating and diagnosing conditions,” which published as US Patent Application Publication 2005/0159790
U.S. patent application Ser. No. 11/349,020, filed Feb. 7, 2006, which published as US Patent Application Publication 2006/0287677
U.S. patent application Ser. No. 11/465,381, filed Aug. 17, 2006, which published as US Patent Application Publication 2007/0083245
U.S. patent application Ser. No. 11/668,305, filed Jan. 19, 2007, which published as US Patent Application Publication 2008/0033509
U.S. patent application Ser. No. 11/874,529, filed Oct. 18, 2007

In an embodiment of the present invention, electrical stimulation system 20 comprises circuitry described in one or more of the above-mentioned applications.

In an embodiment of the present invention, an MTS is stimulated using the magnetic stimulation apparatus and methods described in the above-mentioned U.S. patent application Ser. No. 10/783,113.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. For example, elements which are shown in a figure to be housed within one integral unit may, for some applications, be disposed in a plurality of distinct units. Similarly, apparatus for communication and power transmission which are shown to be coupled in a wireless fashion may, alternatively, be coupled in a wired fashion, and apparatus for communication and power transmission which are shown to be coupled in a wired fashion may, alternatively, be coupled in a wireless fashion.

1. A method comprising:
identifying an electrical stimulation protocol as being suitable for augmenting genesis of one or more cell populations in at least one brain region of the subject; and
augmenting the cell genesis by applying the identified stimulation protocol to a site of the subject selected from the group consisting of: a sphenopalatine ganglion (SPG), a greater palatine nerve, a branch of the greater palatine nerve, a lesser palatine nerve, a sphenopalatine nerve, a communicating branch between a maxillary nerve and an SPG, an otic ganglion, an afferent fiber going into the otic ganglion, an efferent fiber going out of the otic ganglion, an infraorbital nerve, a vidian nerve, a greater superficial petrosal nerve, a lesser deep petrosal nerve, a maxillary nerve, a branch of the maxillary nerve, a nasopalatine nerve, a peripheral site that provides direct or indirect afferent innervation to the SPG, and a peripheral site that is directly or indirectly efferently innervated by the SPG.

2. The method according to claim 1, wherein the site is selected from the group consisting of: the SPG, the greater palatine nerve, the lesser palatine nerve, the sphenopalatine nerve, the communicating branch between the maxillary nerve and the SPG, the otic ganglion, the afferent fiber going into the otic ganglion, the efferent fiber going out of the otic ganglion, the infraorbital nerve, the vidian nerve, the greater superficial petrosal nerve, and the lesser deep petrosal nerve.

3. The method according to claim 1, wherein identifying the stimulation protocol comprises identifying the stimulation protocol as being suitable for augmenting neurogenesis in the at least one brain region.

4. The method according to claim 1, wherein identifying the stimulation protocol comprises identifying the stimulation protocol as being suitable for augmenting angiogenesis in the at least one brain region.

5. The method according to claim 1, wherein identifying the stimulation protocol comprises identifying the stimulation protocol as being suitable for augmenting glia-genesis in the at least one brain region.

6. The method according to claim 1, and comprising identifying that the subject suffers from an adverse cerebral condition, wherein augmenting the cell genesis comprises augmenting the cell genesis responsively to identifying that the subject suffers from the adverse cerebral condition.

7. The method according to claim 6, wherein the cerebral condition causes a brain area of the subject to be diseased, and wherein the at least one brain region is selected from the group consisting of: a vicinity of the diseased brain area, and a brain area other than the vicinity of the diseased brain area.

8. The method according to claim 6, wherein identifying that the subject suffers from the adverse cerebral condition comprises identifying that the subject suffers from a cerebrovascular infarction.

9. The method according to claim 8, wherein augmenting the cell genesis comprising commencing applying the stimulation at least 18 hours after an occurrence of the infarction.

10. The method according to claim 1, and comprising identifying that the subject may benefit from the augmented cell genesis, wherein augmenting the cell genesis comprises augmenting the cell genesis responsively to identifying that the subject may benefit from the augmented cell genesis.

11. A method for treating a subject, comprising:
identifying an electrical stimulation protocol as being suitable for improving a metabolic state of a brain area of a subject; and
improving the metabolic state by applying the stimulation protocol to a site of the subject selected from the group consisting of: a sphenopalatine ganglion (SPG), a greater palatine nerve, a branch of the greater palatine nerve, a lesser palatine nerve, a sphenopalatine nerve, a communicating branch between a maxillary nerve and an SPG, an otic ganglion, an afferent fiber going into the otic ganglion, an efferent fiber going out of the otic ganglion, an infraorbital nerve, a vidian nerve, a greater superficial petrosal nerve, a lesser deep petrosal nerve, a maxillary nerve, a branch of the maxillary nerve, a nasopalatine nerve, a peripheral site that provides direct or indirect afferent innervation to the SPG, and a peripheral site that is directly or indirectly efferently innervated by the SPG.

12. The method according to claim 11, wherein the site is selected from the group consisting of: the SPG, the greater palatine nerve, the lesser palatine nerve, the sphenopalatine nerve, the communicating branch between the maxillary nerve and the SPG, the otic ganglion, the afferent fiber going into the otic ganglion, the efferent fiber going out of the otic ganglion, the infraorbital nerve, the vidian nerve, the greater superficial petrosal nerve, and the lesser deep petrosal nerve.

13. The method according to claim 11, wherein identifying the stimulation protocol comprises identifying the stimulation protocol as suitable for reducing a lactate concentration in the brain area.

14. The method according to claim 11, and comprising identifying that the subject suffers from an adverse cerebral condition, wherein improving the metabolic state comprises improving the metabolic state responsively to identifying that the subject suffers from the adverse cerebral condition.

15. The method according to claim 14, wherein identifying that the subject suffers from the adverse cerebral condition comprises identifying that the subject suffers from a cerebrovascular infarction.

16. The method according to claim 14, wherein improving the metabolic state comprises commencing applying the stimulation at least 18 hours after an occurrence of the infarction.

17. The method according to claim 14, wherein the brain area is an ischemic core of the infarction.

18. The method according to claim 17, wherein identifying the stimulation protocol comprises identifying the stimulation protocol as suitable for reviving at least a portion of the ischemic core.

19. The method according to claim 14, wherein the brain area is an ischemic penumbra of the infarction.

20. The method according to claim 19, wherein identifying the stimulation protocol comprises identifying the stimulation protocol as suitable for reviving at least a portion of the ischemic penumbra.

21. The method according to claim 11, and comprising identifying that the subject may benefit from the improved metabolic state, wherein improving the metabolic state comprises improving the metabolic state responsively to identifying that the subject may benefit from the improved metabolic state.
